(19) United States
(12) Patent Application Publication
(10) Pub. No.: US 2026/0306063 A1
(43) Pub. Date: Oct. 8, 2026

(54) ULTRASOUND CONTRAST IMAGING METHOD, APPARATUS, SYSTEM AND DEVICE, AND STORAGE MEDIUM

(71) Applicant: SONOSCAPE MEDICAL CORP., Shenzhen, Guangdong (CN)

(72) Inventors: Jianwu ZHU, Shenzhen, Guangdong (CN); Wangfeng LIU, Shenzhen, Guangdong (CN); Naizhang FENG, Shenzhen, Guangdong (CN)

(73) Assignee: SONOSCAPE MEDICAL CORP., Shenzhen, Guangdong (CN)

(21) Appl. No.: 18/879,278

(22) PCT Filed: Mar. 28, 2023
(86) PCT No.: PCT/CN2023/084252

§ 371 Date: Dec. 27, 2024

(30) Foreign Application Priority Data
Jun. 27, 2022 (CN) 202210736704.X

(51) Int. Cl. A61B 8/00

(52) U.S. Cl. CPC A61B 8/481; A61B 8/4483; A61B 8/5207; A61B 8/58

(57) ABSTRACT

Provided are an ultrasound contrast imaging method, apparatus, system and device, and a storage medium. The method comprises: determining a plurality of transmission frequencies within an effective bandwidth range of an ultrasonic probe, and generating, on the basis of the plurality of transmission frequencies, a pulse sequence comprising a plurality of ultrasonic signal groups, each of the ultrasonic signal groups comprising a plurality of ultrasonic signals, and each of the ultrasonic signals at least comprising two ultrasonic sub-signals with different periodic transmission frequencies; sequentially transmitting different ultrasonic signals in different ultrasonic signal groups in the pulse sequence, and receiving echo signals generated by contrast agent microbubbles on the transmitted ultrasonic signals, the echo signals comprising fundamental signals and harmonic signals corresponding to different imaging frequencies; processing the echo signals to obtain a plurality of contrast signals corresponding to different imaging frequencies, and generating corresponding contrast images on the basis of the contrast signals; and fusing the contrast images to obtain a target contrast image. The present application meets the requirements of penetrating power, sensitivity, and resolution at the same time by means of one-time contrast imaging.

This application claims priority to Chinese Patent Application No. 202210736704.X, titled “ULTRASOUND CONTRAST IMAGING METHOD, APPARATUS, SYSTEM AND DEVICE, AND STORAGE MEDIUM”, filed on Jun. 27, 2022 with the China National Intellectual Property Administration (CNIPA), which is incorporated herein by reference in its entirety.

### FIELD

The present disclosure relates to the technical field of ultrasonic imaging, and more specifically, to a method and an apparatus for ultrasonic contrast imaging, an ultrasonic imaging system, an ultrasonic device, and a computer-readable storage medium.

### BACKGROUND

In conventional systems for ultrasonic contrast imaging from various manufacturers, contrast images are provided with a penetration/sensitivity mode Pen, a general mode Gen and a resolution mode Res. Users can only choose an imaging mode from the imaging modes provided by the manufacturers. However, attenuation of ultrasound is different for people due to different physical conditions. Therefore, for different characteristics of lesions, such as a depth and a size of the lesion, or multiple lesions in different shapes at the same section, the doctor may first choose an imaging mode based on experience. If a contrast image does not meet an expectation, such as having an insufficient resolution, an insufficient sensitivity, or an insufficient penetration, the doctor may choose another imaging mode to perform the contrast imaging at the second time. Especially when both a tiny lesion and a deeper lesion are on the same section, contrast imaging may be performed many times to achieve good contrast perfusion imaging effect to observe the multiple lesions. Hence, for some special scenarios, requirements of penetration, sensitivity and resolution cannot be met through one-time contrast imaging, which results in increased time costs in clinic and increased cost for contrast agent.

Therefore, how to meet requirements of penetration, sensitivity and resolution through one-time contrast imaging is a technical problem to be solved by those skilled in the art.

### SUMMARY

An objective of the present disclosure is to provide a method and an apparatus for ultrasonic contrast imaging, an ultrasonic device, and a computer-readable storage medium, with which requirements of penetration, sensitivity, and resolution is met through one-time contrast imaging.

To achieve the above objective, a method for ultrasonic contrast imaging is provided in the present disclosure. The method includes:
determining multiple transmission frequencies within an effective bandwidth range of an ultrasonic probe, and generating a pulse sequence including multiple ultrasonic signal groups based on the multiple transmission frequencies, where each of the ultrasonic signal groups includes multiple ultrasonic signals, and each of the ultrasonic signals includes at least two periods of ultrasonic sub-signals having different transmission frequencies, the multiple ultrasonic signals in a same ultrasonic signal group have a same transmission frequency in a corresponding period, and the multiple ultrasonic signals in different ultrasonic signal groups have different transmission frequencies in the corresponding period;
transmitting the multiple ultrasonic signals in the multiple ultrasonic signal groups in the pulse sequence sequentially, and receiving echo signals generated by contrast agent microbubbles in response to the transmitted ultrasonic signals, where the echo signals include fundamental signals and harmonic signals corresponding to different imaging frequencies;
processing the echo signals to obtain multiple contrast signals corresponding to different imaging frequencies, and generating contrast images based on the contrast signals; and
fusing the contrast images to obtain target contrast images.

In an embodiment, the determining multiple transmission frequencies within an effective bandwidth range of an ultrasonic probe, and generating a pulse sequence including multiple ultrasonic signal groups based on the transmission frequencies includes:
determining a reference transmission frequency and a target coefficient, determining a product of the reference transmission frequency and the target coefficient as a first transmission frequency, determining twice the first transmission frequency as a second transmission frequency, determining three times the first transmission frequency as a third transmission frequency, and determining four times the first transmission frequency as a fourth transmission frequency, where the first transmission frequency, the second transmission frequency, the third transmission frequency and the fourth transmission frequency are all within the effective bandwidth range of the ultrasonic probe; and
generating the pulse sequence including a first ultrasonic signal group and a second ultrasonic signal group, where the first ultrasonic signal group is generated based on the first transmission frequency and the second transmission frequency, and the second ultrasonic signal group is generated based on the third transmission frequency and the fourth transmission frequency.

In an embodiment, the determining multiple transmission frequencies within an effective bandwidth range of an ultrasonic probe, and generating a pulse sequence including multiple ultrasonic signal groups based on the transmission frequencies includes:
determining a reference transmission frequency and a target coefficient, determining a product of the reference transmission frequency and the target coefficient as a first transmission frequency, determining twice the first transmission frequency as a second transmission frequency, determining three times the first transmission frequency as a third transmission frequency, determining four times the first transmission frequency as a fourth transmission frequency, and determining a transmission frequency adjacent to the second transmission as a fifth transmission frequency, where the second transmission frequency, the third transmission frequency, the fourth transmission frequency and the fifth transmission frequency are all within the effective bandwidth range of the ultrasonic probe; and
generating the pulse sequence including a first ultrasonic signal group and a second ultrasonic signal group, where the first ultrasonic signal group is generated based on the fifth transmission frequency and the second transmission frequency, and the second ultrasonic signal group is generated based on the third transmission frequency and the fourth transmission frequency.

In an embodiment, the transmitting the multiple ultrasonic signals in the multiple ultrasonic signal groups in the pulse sequence sequentially includes:
determining different voltage amplitudes for ultrasonic sub-signals in the corresponding period in the multiple ultrasonic signals of the same ultrasonic signal group;
determining a same number of array elements of a transmit aperture for ultrasonic sub-signals in the multiple ultrasonic signals of the same ultrasonic signal group; and
transmitting the multiple ultrasonic signals in the multiple ultrasonic signal groups in the pulse sequence sequentially, based on the transmission frequencies, the voltage amplitudes and the numbers of array elements of the transmitting aperture for different ultrasonic sub-signals in the multiple ultrasonic signals.

In an embodiment, each of the ultrasonic signal groups includes a first ultrasonic signal and a second ultrasonic signal, and the determining different voltage amplitudes for ultrasonic sub-signals in the corresponding period in the multiple ultrasonic signals of the same ultrasonic signal group includes:
determining a first voltage amplitude for an ultrasonic sub-signal in the first ultrasonic signal, and determining a second voltage amplitude for an ultrasonic sub-signal in the second ultrasonic signal, where the second voltage amplitude is a preset multiple of the first voltage amplitude.

In an embodiment, the second voltage amplitude is twice the first voltage amplitude.

In an embodiment, each of the ultrasonic signal groups includes a first ultrasonic signal, a second ultrasonic signal and a third ultrasonic signal, and the determining different voltage amplitudes for ultrasonic sub-signals in the corresponding period in the multiple ultrasonic signals of the same ultrasonic signal group includes:
determining a third voltage amplitude for an ultrasonic sub-signal in the first ultrasonic signal and an ultrasonic sub-signal in the third ultrasonic signal, and determining a fourth voltage amplitude for an ultrasonic sub-signal in the second ultrasonic signal, where the fourth voltage amplitude is a preset multiple of the third voltage amplitude.

In an embodiment, the fourth voltage amplitude is twice the third voltage amplitude.

In an embodiment, the transmitting the multiple ultrasonic signals in the multiple ultrasonic signal groups in the pulse sequence sequentially includes:
determining a same voltage amplitude for the ultrasonic sub-signals in the corresponding period in the multiple ultrasonic signals of the same ultrasonic signal group;
determining different numbers of array elements of the transmit aperture for the ultrasonic sub-signals in the multiple ultrasonic signals of the same ultrasonic signal group; and
transmitting the multiple ultrasonic signals in the multiple ultrasonic signal groups in the pulse sequence sequentially, based on transmission frequencies, voltage amplitudes and the numbers of array elements of the transmit aperture for different ultrasonic sub-signals in the multiple ultrasonic signals.

In an embodiment, each of the ultrasonic signal groups includes a first ultrasonic signal and a second ultrasonic signal, and the determining different numbers of array elements of the transmitting aperture for the ultrasonic sub-signals in the multiple ultrasonic signals of the same ultrasonic signal group includes:
determining a first number of array elements of the transmitting aperture for the ultrasonic sub-signals in the first ultrasonic signal, and determining a second number of array elements of the transmitting aperture for the ultrasonic sub-signals in the second ultrasonic signal, where the second number of array elements of the transmitting aperture is a preset multiple of the first number of array elements of the transmitting aperture.

In an embodiment, the second number of array elements of the transmitting aperture is twice the first number of array elements of the transmitting aperture.

In an embodiment, each of the ultrasonic signal groups includes a first ultrasonic signal, a second ultrasonic signal and a third ultrasonic signal, and the determining different numbers of array elements of the transmitting aperture for the ultrasonic sub-signals in the multiple ultrasonic signals of the same ultrasonic signal group includes:
determining the transmit aperture of the ultrasonic sub-signal in the first ultrasonic signal one of an odd aperture and an even aperture, determining the transmit aperture of the ultrasonic sub-signal in the second ultrasonic signal as a full aperture, and determining the transmit aperture of the ultrasonic sub-signal in the third ultrasonic signal is the other one of the odd aperture and the even aperture.

In an embodiment, the processing the echo signals to obtain multiple contrast signals corresponding to different imaging frequencies includes:
performing weighted processing on the echo signals of the multiple ultrasonic signals in each of the ultrasonic signal groups, based on a voltage amplitude relationship and a phase relationship among the multiple ultrasonic signals in each of the ultrasonic signal groups, to obtain the contrast signals corresponding to each of the ultrasonic signal groups; and
demodulating the contrast signals corresponding to each of the ultrasonic signal groups by using demodulation circuits corresponding to the different imaging frequencies, to obtain the contrast signals corresponding to the different imaging frequencies.

In an embodiment, the performing weighted processing on the echo signals of the multiple ultrasonic signals in each of the ultrasonic signal groups, based on the voltage amplitude relationship and the phase relationship among the multiple ultrasonic signals in each of the ultrasonic signal groups, to obtain contrast signals corresponding to each of the ultrasonic signal groups, includes:
determining weighting coefficients of the echo signals, based on the voltage amplitude relationship among the multiple ultrasonic signals in each of the ultrasonic signal groups;
determining an operational relationship of the echo signals based on the phase relationship among the multiple ultrasonic signals in each of the ultrasonic signal groups, where the operational relationship is determined as a summation in response to the phase relationship being out of phase, and the operational relationship is determined as a subtraction in response to the phase relationship being in phase; and
performing weighted processing on the echo signals of the multiple ultrasonic signals in each of the ultrasonic signal groups based on the weighting coefficients and the operational relationship, to obtain the contrast signals corresponding to each of the ultrasonic signal groups.

In an embodiment, the transmitting the multiple ultrasonic signals in the multiple ultrasonic signal groups in the pulse sequence sequentially and receiving echo signals generated by contrast agent microbubbles in response to the transmitted ultrasonic signals includes:
transmitting the multiple ultrasonic signals in the multiple ultrasonic signal groups in the pulse sequence sequentially, where in response the multiple ultrasonic signals, the contrast agent microbubbles generate fundamental signals corresponding to different first imaging frequencies, generate a multi-harmonic signal corresponding to a second imaging frequency based on the fundamental signals corresponding to the first imaging frequencies, and generate a differential harmonic signal corresponding to a third imaging frequency based on the fundamental signals corresponding to the different first imaging frequencies, where the second imaging frequency is an integer multiple of the first imaging frequency, and the third imaging frequency is at least one of a difference between first imaging frequencies or a sum of first imaging frequencies; and
receiving the fundamental signals corresponding to the first imaging frequencies, the multi-harmonic signal corresponding to the second imaging frequency, and the differential harmonic signal corresponding to the third imaging frequency.

In an embodiment, the fusing the contrast images to obtain the target contrast images includes:
determining weighting coefficients of contrast images corresponding to different imaging modes, and performing weighted fusion on the contrast images based on the weighting coefficients of the contrast images, to obtain the target contrast images corresponding to different imaging modes.

In an embodiment, the imaging modes include a penetration mode and/or a resolution mode, the weighting coefficient of the contrast image corresponding to the penetration mode is negatively correlated with an imaging frequency corresponding to the contrast image, and the weighting coefficient of the contrast image corresponding to the resolution mode is positively correlated with the imaging frequency corresponding to the contrast image.

In an embodiment, the determining weighting coefficients of the contrast images corresponding to the different imaging modes includes:
displaying, for one target imaging mode, an adjustment window corresponding to the target contrast image, where the adjustment window includes adjustment areas for the contrast images; and
receiving an adjustment instruction on at least one of the adjustment areas, and adjusting a weighting coefficient of the contrast image corresponding to the at least one of the adjustment areas based on the adjustment instruction, to obtain the weighting coefficients of the contrast images in the target imaging mode.

To achieve the above objective, an apparatus for ultrasonic contrast imaging is provided in the present disclosure. The apparatus includes: a generation module, a transmission module, a processing module and a fusion module.

The generation module is configured to determine multiple transmission frequencies within an effective bandwidth range of an ultrasonic probe, and generate a pulse sequence including multiple ultrasonic signal groups based on the transmission frequencies, where each of the ultrasonic signal groups includes multiple ultrasonic signals, and each of the ultrasonic signals includes at least two periods of ultrasonic sub-signals having different transmission frequencies, the multiple ultrasonic signals in a same ultrasonic signal group have a same transmission frequency in a corresponding period, and the multiple ultrasonic signals in different ultrasonic signal groups have different transmission frequencies in the corresponding period.

The transmission module is configured to transmit the multiple ultrasonic signals in the multiple ultrasonic signal groups in the pulse sequence sequentially, and receive echo signals generated by contrast agent microbubbles in response to the transmitted ultrasonic signals, where the echo signals include fundamental signals and harmonic signals corresponding to different imaging frequencies.

The processing module is configured to process the echo signals to obtain multiple contrast signals corresponding to the different imaging frequencies, and generate contrast images based on the contrast signals.

The fusion module is configured to fuse the contrast images to obtain target contrast images.

To achieve the above objective, an ultrasonic imaging system is provided in the present disclosure. The ultrasonic imaging system includes an ultrasonic host, an ultrasonic probe, and a master computer. The ultrasonic host is communicatively connected to the ultrasonic probe and the master computer.

The ultrasonic host is configured to perform the method for ultrasonic contrast imaging according to any of the above embodiments.

To achieve the above objective, an ultrasonic device is provided in the present disclosure. The ultrasonic device includes: a memory configured to store a computer program; and a processor configured to implement, when executing the computer program, the method for ultrasonic contrast imaging according to any of the above embodiments.

To achieve the above objective, a computer-readable storage medium is provided in the present disclosure. The computer-readable storage medium stores a computer program. The computer program, when executed by a processor, performs the method for ultrasonic contrast imaging according to any of the above embodiments.

From the above solution, the method for ultrasonic contrast imaging is provided in the present disclosure. The method includes: determining multiple transmission frequencies within an effective bandwidth range of an ultrasonic probe, and generating a pulse sequence including multiple ultrasonic signal groups based on the transmission frequencies, where each of the ultrasonic signal groups includes multiple ultrasonic signals, and each of the ultrasonic signals includes at least two periods of ultrasonic sub-signals having different transmission frequencies, the multiple ultrasonic signals in a same ultrasonic signal group have a same transmission frequency in a corresponding period, and the multiple ultrasonic signals in different ultrasonic signal groups have different transmission frequencies in the corresponding period; transmitting the multiple ultrasonic signals in the multiple ultrasonic signal groups in the pulse sequence sequentially, and receiving echo signals generated by contrast agent microbubbles in response to the transmitted ultrasonic signals, where the echo signals include fundamental signals and harmonic signals corresponding to different imaging frequencies; processing the echo signals to obtain multiple contrast signals corresponding to different imaging frequencies, and generating contrast images based on the contrast signals; and fusing the contrast images to obtain a target contrast image.

With the method for ultrasonic contrast imaging according to the present disclosure, the ultrasonic probe can obtain multiple contrast signals corresponding to different imaging frequencies through one-time transmission of a complete pulse sequence. The contrast signals include fundamental signals and harmonic signals of different imaging frequencies. A fundamental signal and a harmonic signal of one imaging frequency enhance each other, to obtain an enhanced contrast signal. The contrast image generated based on the enhanced contrast signal can meet requirements of penetration, sensitivity and resolution. The apparatus for ultrasonic contrast imaging, the ultrasonic imaging system, the ultrasonic device and the computer-readable storage medium provided in the present disclosure can achieve the same technical effects as described above.

It should be understood that the foregoing general description and the following detailed description are exemplary only, and do not limit the present disclosure.

### BRIEF DESCRIPTION OF THE DRAWINGS

For clearer illustration of the technical solutions according to embodiments of the present disclosure or conventional techniques, hereinafter briefly described are the drawings to be applied in embodiments of the present disclosure or conventional techniques. Apparently, the drawings in the following descriptions are only some embodiments of the present disclosure, and other drawings may be obtained by those skilled in the art based on the provided drawings without any creative effort. The accompanying drawings are for providing a further understanding of the present disclosure, and form a part of the specification. The drawings, together with the following embodiments, are for explaining the present disclosure, and do not constitute any limitation to the present disclosure. In the drawings:

FIG. 1 is an architectural diagram of an ultrasonic imaging system according to an exemplary embodiment;

FIG. 2 is an architectural diagram of an ultrasonic imaging system according to another exemplary embodiment;

FIG. 3 is a flow chart of a method for ultrasonic contrast imaging according to an exemplary embodiment;

FIG. 4a is a schematic diagram of a first ultrasonic signal according to an exemplary embodiment;

FIG. 4b is a schematic diagram of a second ultrasonic signal according to an exemplary embodiment;

FIG. 4c is a schematic diagram of a third ultrasonic signal according to an exemplary embodiment;

FIG. 4d is a schematic diagram of a fourth ultrasonic signal according to an exemplary embodiment;

FIG. 5a is a schematic diagram of a first ultrasonic signal according to another exemplary embodiment;

FIG. 5b is a schematic diagram of a second ultrasonic signal according to another exemplary embodiment;

FIG. 5c is a schematic diagram of a third ultrasonic signal according to another exemplary embodiment;

FIG. 5d is a schematic diagram of a fourth ultrasonic signal according to another exemplary embodiment;

FIG. 6a is a schematic diagram of a first ultrasonic signal according to yet another exemplary embodiment;

FIG. 6b is a schematic diagram of a second ultrasonic signal according to yet another exemplary embodiment;

FIG. 6c is a schematic diagram of a third ultrasonic signal according to yet another exemplary embodiment;

FIG. 6d is a schematic diagram of a fourth ultrasonic signal according to yet another exemplary embodiment;

FIG. 7a is a schematic diagram of a first ultrasonic signal according to yet another exemplary embodiment;

FIG. 7b is a schematic diagram of a second ultrasonic signal according to yet another exemplary embodiment;

FIG. 7c is a schematic diagram of a third ultrasonic signal according to yet another exemplary embodiment;

FIG. 7d is a schematic diagram of a fourth ultrasonic signal according to yet another exemplary embodiment;

FIG. 7e is a schematic diagram of a fifth ultrasonic signal according to an exemplary embodiment;

FIG. 7f is a schematic diagram of a sixth ultrasonic signal according to an exemplary embodiment;

FIG. 8 is a schematic display diagram of an ultrasonic contrast image according to an exemplary embodiment;

FIG. 9 is a schematic diagram of an adjustment frame for a target contrast image according to an exemplary embodiment;

FIG. 10 is a structural diagram of an apparatus for ultrasonic contrast imaging according to an exemplary embodiment; and

FIG. 11 is a structural diagram of an ultrasonic device according to an exemplary embodiment.

### DETAILED DESCRIPTION

Hereinafter technical solutions of embodiments of the present disclosure are described clearly and completely in conjunction with the drawings of the embodiments of the present disclosure. Apparently, the embodiments described below are only some embodiments, rather than all the embodiments of the present disclosure. Any other embodiments obtained by those skilled in the art based on the embodiments in the present disclosure without any creative effort shall fall within the protection scope of the present disclosure. Moreover, in the embodiments of the present disclosure, words “first”, “second”, and the like, are used to distinguish similar objects from each other, rather than to describe a specific sequence or order.

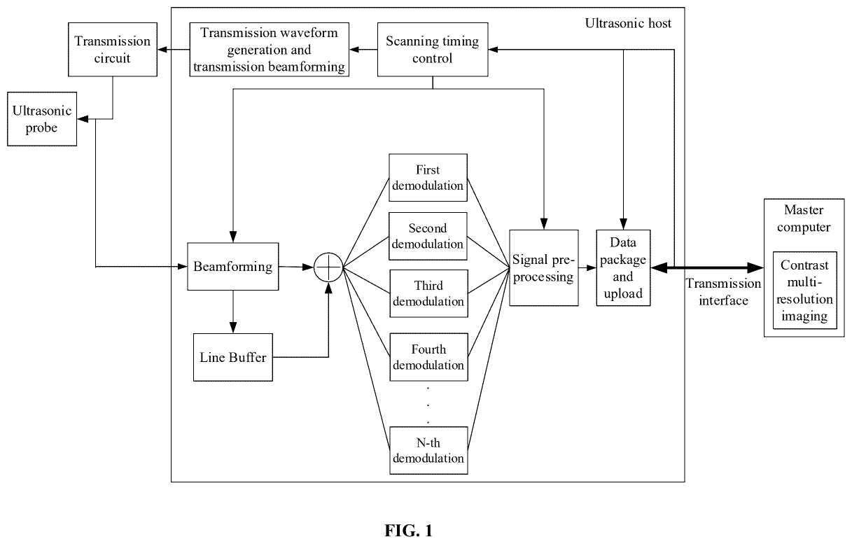

The present disclosure is applicable to an ultrasonic imaging system as shown in FIG. 1. The ultrasonic imaging system includes an ultrasonic host, an ultrasonic probe, and a master computer. The ultrasonic host determines multiple transmission frequencies within an effective bandwidth range of the ultrasonic probe, and generates a pulse sequence, that is, a transmitting waveform, including multiple ultrasonic signal groups by controlling a scanning timing. Each of the ultrasonic signal groups includes multiple ultrasonic signals. Then, transmitting beams are obtained from synthesis. The transmitting beams are transmitted from a transmission circuit to the ultrasonic probe, and then transmitted to a to-be-inspected tissue through the ultrasonic probe. Contrast agent microbubbles in the tissue generate echo signals. The ultrasonic host performs beamforming on the echo signals of the different ultrasonic signals and stores in a linebuffer. In the linebuffer, the echo signals of the different ultrasonic signals are processed by weighted summation or difference based on weights, obtaining contrast signals corresponding to different ultrasonic signal groups. The linebuffer may be a Block random access memory (RAM) resource in a field programmable gate array (FPGA), or may be an external memory, such as a double data rate (DDR) synchronous dynamic random memory, or a hard disk, which is not specifically limited here. Further, the ultrasonic host demodulates the contrast signals corresponding to the ultrasonic signal groups through a demodulation circuit (such as the N-channel demodulation circuit in FIG. 1, where N is a natural number greater than 0), to obtain contrast signals corresponding to different imaging frequencies. After signal pre-processing, data is packaged and uploaded and sent to the master computer through a transmission interface, so that the master computer performs multi-resolution contrast ultrasound imaging. In an implementation, the master computer performs weighted fusion of different contrast images based on weighting coefficients of contrast images corresponding to different imaging modes, to obtain target contrast images corresponding to different imaging modes, and then displays the target contrast images on a display device.

As can be seen from FIG. 1, the weighting, demodulation and signal processing of the echo signals of different ultrasonic signals are performed at a front end of an ultrasonic device.

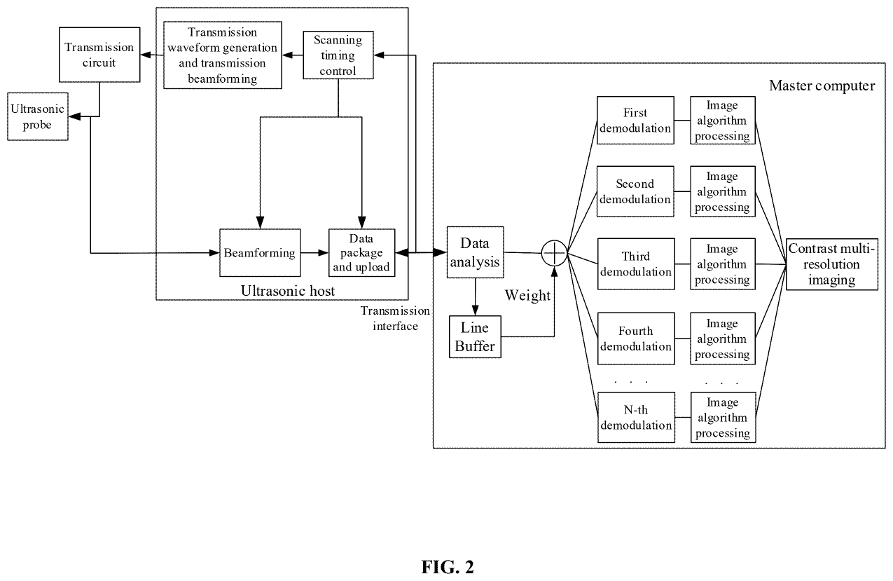

Apparently, the weighting, demodulation and signal processing of the echo signals of different ultrasonic signals may be performed at a rear end, i.e., the master computer. A specific architecture diagram is shown in FIG. 2. FIG. 2 is different from FIG. 1 in that: after the ultrasonic host performs beamforming on the echo signals of different ultrasonic signals, data is packaged and uploaded and sent to the master computer through the transmission interface. The master computer performs data analysis and then stores the analyzed data in the linebuffer. In the linebuffer, the echo signals of different ultrasonic signals are processed by weighted summation or difference based on weights, obtaining contrast signals corresponding to different ultrasonic signal groups. Further, the contrast signals corresponding to the ultrasonic signal groups are demodulated through a demodulation circuit (such as the N-channel demodulation circuit in FIG. 2, where N is a natural number greater than 0), to obtain contrast signals corresponding to different imaging frequencies. Then, the contrast signals are processed through an imaging algorithm to obtain contrast images corresponding to different imaging frequencies. Then, the master computer implements multi-resolution contrast ultrasound imaging.

A method for ultrasonic contrast imaging is disclosed in an embodiment of the present disclosure, with which requirements of penetration, sensitivity, and resolution is met through one-time contrast imaging.

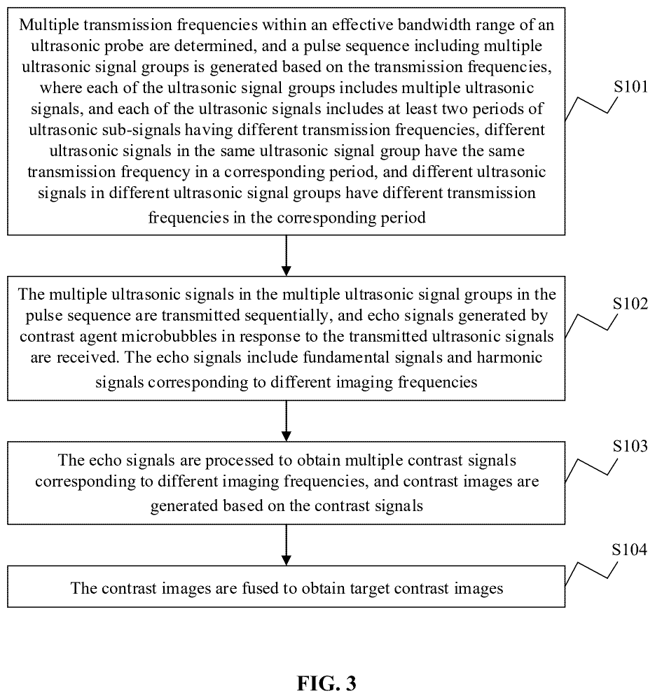

Referring to FIG. 3, a flow chart of a method for ultrasonic contrast imaging is shown according to an exemplary embodiment. As shown in FIG. 3, the method includes steps S101 to S104.

In S101, multiple transmission frequencies within an effective bandwidth range of an ultrasonic probe are determined, and a pulse sequence including multiple ultrasonic signal groups is generated based on the transmission frequencies. Each of the ultrasonic signal groups includes multiple ultrasonic signals, and each of the ultrasonic signals includes at least two periods of ultrasonic sub-signals having different transmission frequencies. Different ultrasonic signals in the same ultrasonic signal group have the same transmission frequency in a corresponding period, and different ultrasonic signals in different ultrasonic signal groups have different transmission frequencies in the corresponding period.

In a specific implementation, the multiple transmission frequencies are determined within the effective bandwidth range of the ultrasonic probe. The effective bandwidth range refers to a frequency range of the ultrasound which can be transmitted by the ultrasonic probe. It should be noted that the multiple transmission frequencies cover the effective bandwidth range of the ultrasonic probe as much as possible. That is, the minimum one of the transmission frequencies is close to the minimum effective bandwidth of the ultrasonic probe, that is, the minimum frequency of the ultrasound which can be transmitted by the ultrasonic probe; and the maximum one of the transmission frequencies is close to the maximum effective bandwidth of the ultrasonic probe, that is, the maximum frequency of the ultrasound which can be transmitted by the ultrasonic probe.

Further, the pulse sequence, including ultrasonic signals transmitted by the ultrasonic probe, is generated based on the multiple determined transmission frequencies. The pulse sequence includes multiple ultrasonic signal groups, and each of the ultrasonic signals includes at least two periods of ultrasonic sub-signals having different transmission frequencies. Different ultrasonic sub-signals in the same ultrasonic signal may have the same or different voltage amplitudes (unless otherwise specified, the same voltage amplitude is illustrated in the embodiments of the present disclosure as an example). Different ultrasonic signals in the same ultrasonic signal group have the same transmission frequency in the corresponding period, and different ultrasonic signals in different ultrasonic signal groups have different transmission frequencies in the corresponding period. In an embodiment, the two periods may include a first period and a second period. Further, the ultrasonic signal includes an ultrasonic sub-signal in the first period and an ultrasonic sub-signal in the second period, and the ultrasonic sub-signal in the first period has a different transmission frequency from the ultrasonic sub-signal in the second period.

As a preferred implementation, step S101 may include: determining a reference transmission frequency and a target coefficient, determining a product of the reference transmission frequency and the target coefficient as a first transmission frequency, determining twice the first transmission frequency as a second transmission frequency, determining three times the first transmission frequency as a third transmission frequency, and determining four times the first transmission frequency as a fourth transmission frequency, where the first transmission frequency, the second transmission frequency, the third transmission frequency and the fourth transmission frequency are all within the effective bandwidth range of the ultrasonic probe; and generating the pulse sequence including a first ultrasonic signal group and a second ultrasonic signal group, where the first ultrasonic signal group is generated based on the first transmission frequency and the second transmission frequency, and the second ultrasonic signal group is generated based on the third transmission frequency and the fourth transmission frequency. In an embodiment, an input command of the target coefficient may be received, and a value of the target coefficient is determined based on a value carried in the input command. The target coefficient may be a value between 0 and 1. In an embodiment, the reference transmission frequency may refer to a basic transmission frequency for transmitting the ultrasonic pulse sequence, which may be understood as a unit of a transmission frequency. The reference transmission frequency may be a preset parameter of the ultrasonic imaging system. In an implementation, an input command of the reference transmission frequency may be received, and a value of the reference transmission frequency may be determined based on a value carried in the input command.

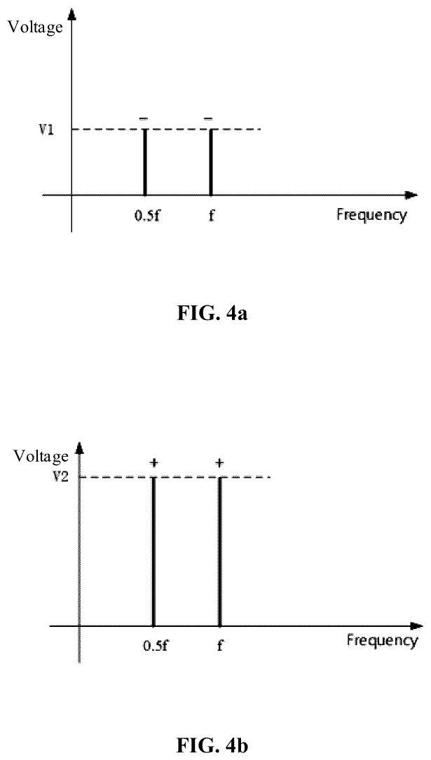

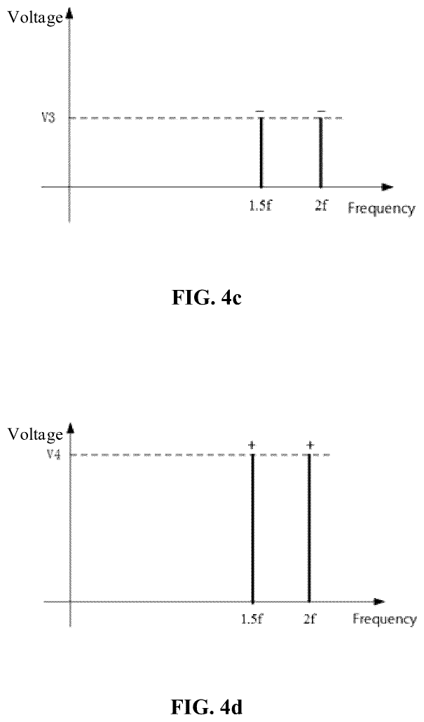

It can be understood that for a general ultrasonic probe, four transmission frequencies can cover the effective bandwidth range of the ultrasonic probe. In an embodiment, it is assumed that the reference transmission frequency is f and the target coefficient may be 0.5. In this case, the first transmission frequency is 0.5f, the second transmission frequency is f, the third transmission frequency is 1.5f, and the fourth transmission frequency is 2f. If the first ultrasonic signal in the first ultrasonic signal group has a voltage amplitude V1 and a phase − (that is, a negative phase), the second ultrasonic signal in the first ultrasonic signal group has a voltage amplitude V2 and a phase + (that is, a positive phase), the third ultrasonic signal in the second ultrasonic signal group has a voltage amplitude V3 and a phase −, and the fourth ultrasonic signal in the second ultrasonic signal group has a voltage amplitude V4 and a phase +, then the first ultrasonic signal in the first ultrasonic signal group is as shown in FIG. 4a (in which the abscissa Frequency represents a frequency, and the ordinate represents a voltage), the second ultrasonic signal in the first ultrasonic signal group is as shown in FIG. 4b, the third ultrasonic signal in the second ultrasonic signal group is as shown in FIG. 4c, and the fourth ultrasonic signal in the second ultrasonic signal group is as shown in FIG. 4d. For example, for an ultrasonic probe having a center frequency of 3.375 MHz and an effective bandwidth of 120%, the effective bandwidth range is from 1.35 MHz to 5.4 MHz. Therefore, the reference transmission frequency f may be determined as 2.7 MHz, and the target coefficient may be 0.5. Then, the four transmission frequencies are 1.35 MHz, 2.7 Mhz, 4.05 MHz, and 5.4 MHz, which correspond to 0.5f, f, 1.5f, and 2f, respectively, covering the effective bandwidth range of the ultrasonic probe.

As a preferred implementation, the step S101 may include: determining a reference transmission frequency and a target coefficient, determining a product of the reference transmission frequency and the target coefficient as a first transmission frequency, determining twice the first transmission frequency as a second transmission frequency, determining three times the first transmission frequency as a third transmission frequency, determining four times the first transmission frequency as a fourth transmission frequency, and determining a transmission frequency adjacent to the second transmission as a fifth transmission frequency, where the second transmission frequency, the third transmission frequency, the fourth transmission frequency and the fifth transmission frequency are all within the effective bandwidth range of the ultrasonic probe; and generating the pulse sequence including a first ultrasonic signal group and a second ultrasonic signal group, where the first ultrasonic signal group is generated based on the fifth transmission frequency and the second transmission frequency, and the second ultrasonic signal group is generated based on the third transmission frequency and the fourth transmission frequency. The fifth transmission frequency may be within a preset range of the second transmission frequency, for example, within a range of 10% greater than or less than the second transmission frequency. In an embodiment, the fifth transmission frequency may be equal to the second transmission frequency. That is, the first ultrasonic signal group is generated based on two second transmission frequencies.

In an embodiment, if the ultrasonic probe has a narrow effective bandwidth range and the reference transmission frequency and the target coefficient are determined, a smaller number of transmission frequencies are determined to cover the effective bandwidth range of the ultrasonic probe.

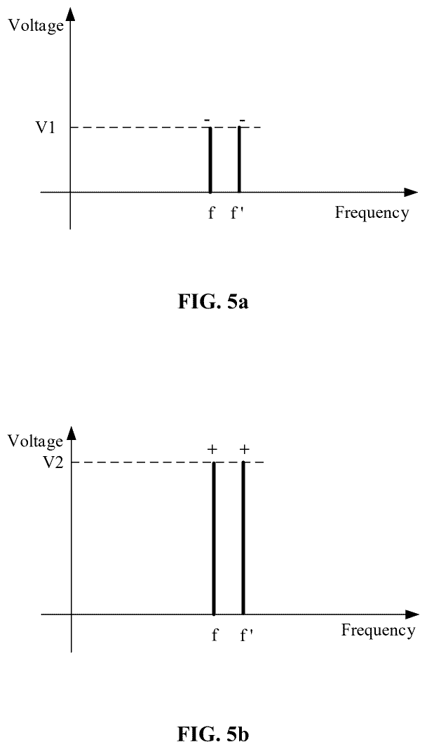

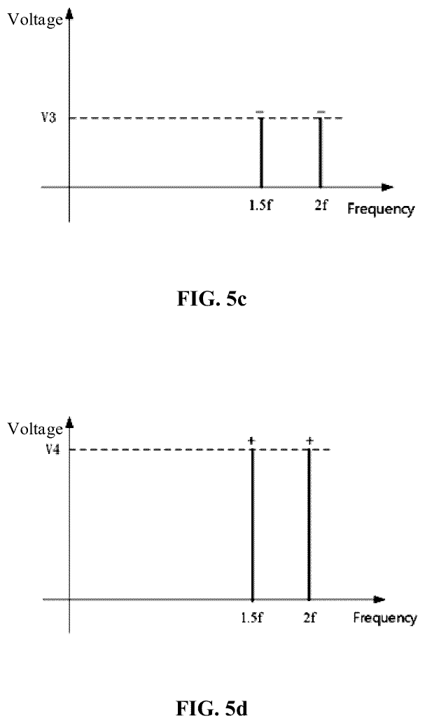

For example, for an ultrasonic probe having a center frequency of 3.0 MHz and an effective bandwidth of 70%, the effective bandwidth range is from 1.95 MHz to 4.05 MHz. The reference transmission frequency f is 2.0 Mhz, and the target coefficient is 0.5. There has 0.5f=1.0 Mhz, f=2.0 Mhz, 1.5f=3.0 Mhz, 2f=4.0 Mhz. The 0.5f exceeds the effective bandwidth range of the ultrasonic probe, and the three transmission frequencies of f, 1.5f, and 2f can be determined to cover the effective bandwidth range of the ultrasonic probe. Hence, the first ultrasonic signal and the second ultrasonic signal each can be configured to include two periods of ultrasonic sub-signals having transmission frequencies of f and f′, respectively. The first ultrasonic signal, the second ultrasonic signal, a third ultrasonic signal and a fourth ultrasonic signal are as shown in FIG. 5a to FIG. 5d, respectively. It should be noted that f′ is within the effective bandwidth range of the ultrasonic probe, and f and f′ may be equal to or different from each other, which is not specifically limited here. In the case of f≠f′, f′ may be determined as frequency adjacent to f or 1.95 MHz.

In S102, the multiple ultrasonic signals in the multiple ultrasonic signal groups in the pulse sequence are transmitted sequentially, and echo signals generated by contrast agent microbubbles in response to the transmitted ultrasonic signals are received. The echo signals include fundamental signals and harmonic signals corresponding to different imaging frequencies.

In this step, the pulse sequence generated in the previous step is transmitted by the ultrasonic probe, that is, the ultrasonic probe transmits the multiple ultrasonic signals in multiple ultrasonic signal groups sequentially. The echo signals generated by contrast agent microbubbles in response to the transmitted ultrasonic signals are received. The imaging frequency mentioned in this step is a frequency of an echo signal.

It can be understood that the multiple ultrasonic signals in the same ultrasonic signal group received by the contrast agent microbubbles may have different voltage amplitudes. That is, the multiple transmitted ultrasonic signals in the same ultrasonic signal group may have different voltage amplitudes. As a feasible implementation, the step S102 may include: determining different voltage amplitudes for ultrasonic sub-signals in the corresponding period in the multiple ultrasonic signals in the same ultrasonic signal group; determining a same number of array elements of a transmitting aperture for ultrasonic sub-signals in the multiple ultrasonic signals in the same ultrasonic signal group; and transmitting the multiple ultrasonic signals in the multiple ultrasonic signal groups in the pulse sequence sequentially, based on transmission frequencies, voltage amplitudes and the numbers of array elements of the transmitting aperture for different ultrasonic sub-signals in the multiple ultrasonic signals. The number of array elements of the transmitting aperture may be adjusted based one setting of the voltage amplitudes. For example, voltage amplitudes of different ultrasonic signals in the same ultrasonic signal group in the corresponding period are different, and the number of array elements of the transmitting aperture is the same when transmitting different ultrasonic signals in the same ultrasonic signal group. Alternatively, voltage amplitudes of different ultrasonic signals in the same ultrasonic signal group in the corresponding period are the same, and the numbers of array elements of the transmitting aperture are different when transmitting different ultrasonic signals in the same ultrasonic signal group.

It should be noted that the number of ultrasonic signals included in each ultrasonic signal group is not limited in the embodiment, and each ultrasonic signal group may include two ultrasonic signals or three ultrasonic signals.

In the case where each ultrasonic signal group includes two ultrasonic signals, i.e., a first ultrasonic signal and a second ultrasonic signal, the determining different voltage amplitudes for ultrasonic sub-signals in the corresponding period in the multiple ultrasonic signals of the same ultrasonic signal group includes: determining a first voltage amplitude for an ultrasonic sub-signal in the first ultrasonic signal, and determining a second voltage amplitude for an ultrasonic sub-signal in the second ultrasonic signal, where the second voltage amplitude is a preset multiple of the first voltage amplitude. In a specific implementation, the voltage amplitude of the ultrasonic sub-signal in the second ultrasonic signal is a preset multiple of the voltage amplitude of the ultrasonic sub-signal in the first ultrasonic signal, and the number of array elements of the transmitting aperture when the ultrasonic sub-signal in the second ultrasonic signal is transmitted is the same as the number of array elements of the transmitting aperture when the ultrasonic sub-signal in the first ultrasonic signal is transmitted. In this way, it can be realized that voltage amplitudes of the first ultrasonic signal and the second ultrasonic signal received by the contrast agent microbubbles are different. In an embodiment, the preset multiple may be a multiple more than 1, that is, the second voltage amplitude is greater than the first voltage amplitude. In addition, the preset multiple may be an integer multiple or a non-integer multiple.

It should be noted that, in order to facilitate subsequent processing of the echo signals, preferably, the second voltage amplitude is twice the first voltage amplitude, that is, the voltage amplitude of the ultrasonic sub-signal in the second ultrasonic signal is twice the voltage amplitude of the ultrasonic sub-signal in the first ultrasonic signal.

For example, the pulse sequence includes two ultrasonic signal groups, i.e., a first ultrasonic signal group and a second ultrasonic signal group. The first ultrasonic signal group includes a first ultrasonic signal and a second ultrasonic signal, and the second ultrasonic signal group includes a third ultrasonic signal and a fourth ultrasonic signal. Each ultrasonic signal includes two periods of ultrasonic sub-signals. The first ultrasonic signal has a voltage amplitude of V1, and includes an ultrasonic sub-signal f1 in a first period and an ultrasonic sub-signal f2 in a second period. The second ultrasonic signal has a voltage amplitude of V2, and includes an ultrasonic sub-signal f1 in the first period and an ultrasonic sub-signal f2 in the second period. The third ultrasonic signal has a voltage amplitude of V3, and includes an ultrasonic sub-signal f3 in the first period and an ultrasonic sub-signal f4 in the second period. The fourth ultrasonic signal has a voltage amplitude of V4, and includes an ultrasonic sub-signal f3 in the first period and an ultrasonic sub-signal f4 in the second period. When V1≠V2 and V3≠V4, the number of array elements of the transmitting aperture is the same when the respective ultrasonic signals are transmitted. Preferably, V2=2×V1 and V4=2×V3.

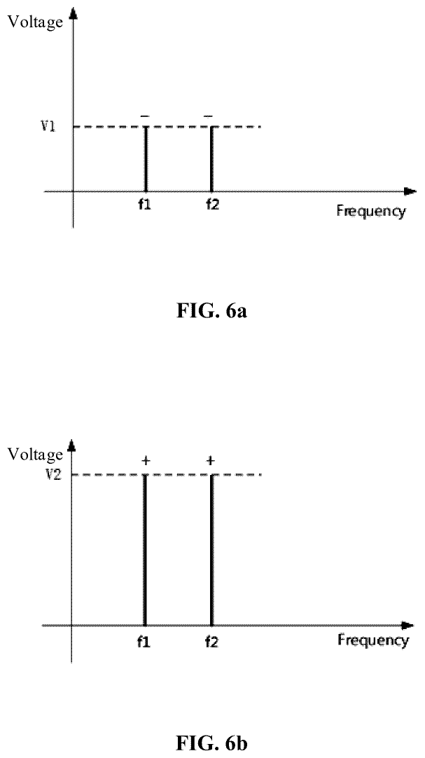

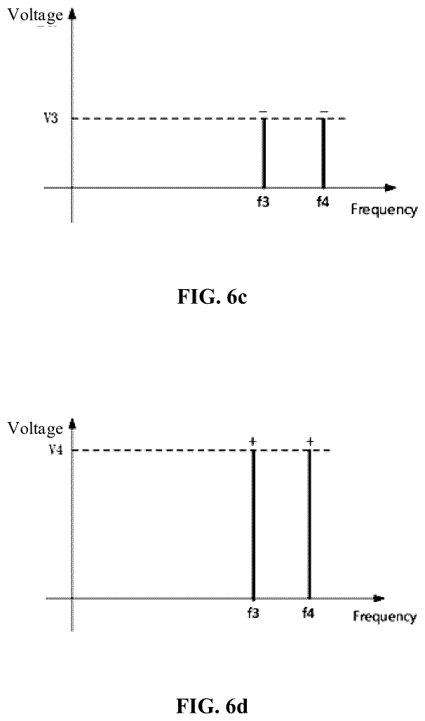

It should be noted that a phase relationship of the multiple ultrasonic signals in the same ultrasonic signal group is not limited in the embodiment. The multiple ultrasonic signals may be in phase or out of phase. That is, the first ultrasonic signal and the second ultrasonic signal in the first ultrasonic signal group may be in phase or out of phase. The third ultrasonic signal and the fourth ultrasonic signal in the second ultrasonic signal group may be in phase or out of phase. For example, the first ultrasonic signal is as shown in FIG. 6a, the second ultrasonic signal is as shown in FIG. 6b, and the first ultrasonic signal and the second ultrasonic signal are out of phase. In addition, the third ultrasonic signal is as shown in FIG. 6c, the fourth ultrasonic signal is as shown in FIG. 6d, and the third ultrasonic signal and the fourth ultrasonic signal are also out of phase.

In the case where each ultrasonic signal group includes three ultrasonic signals, i.e., a first ultrasonic signal, a second ultrasonic signal and a third ultrasonic signal, the determining different voltage amplitudes for ultrasonic sub-signals in the corresponding period in the multiple ultrasonic signals of the same ultrasonic signal group includes: determining a third voltage amplitude for an ultrasonic sub-signal in the first ultrasonic signal and an ultrasonic sub-signal in the third ultrasonic signal, and determining a fourth voltage amplitude for an ultrasonic sub-signal in the second ultrasonic signal, where the fourth voltage amplitude is a preset multiple of the third voltage amplitude. In a specific implementation, the voltage amplitude of the ultrasonic sub-signal in the first ultrasonic signal is the same as the voltage amplitude of the ultrasonic sub-signal in the third ultrasonic signal, and the voltage amplitude of the ultrasonic sub-signal in the second ultrasonic signal is a preset multiple of the voltage amplitude of the ultrasonic sub-signal in the first ultrasonic signal or the third ultrasonic signal. The number of array elements of the transmitting aperture is the same when transmitting the ultrasonic sub-signal in the first ultrasonic signal, the ultrasonic sub-signal in the second ultrasonic signal, and the ultrasonic sub-signal in the third ultrasonic signal. In this way, it can be realized that voltage amplitudes of the first ultrasonic signal and the second ultrasonic signal received by the contrast agent microbubbles are different. In an embodiment, the preset multiple may be a multiple more than 1, that is, the fourth voltage amplitude is greater than the third voltage amplitude, and the voltage amplitude of the ultrasonic sub-signal in the second ultrasonic signal is greater than the voltage amplitude of the ultrasonic sub-signal in the first ultrasonic signal or the third ultrasonic signal. In addition, the preset multiple may be an integer multiple or a non-integer multiple.

It should be noted that, in order to facilitate subsequent processing of the echo signals, preferably, the fourth voltage amplitude is twice the third voltage amplitude. That is, the voltage amplitude of the ultrasonic sub-signal in the second ultrasonic signal is twice the voltage amplitude of the ultrasonic sub-signal in the first ultrasonic signal and the third ultrasonic signal, or, the voltage amplitude of the ultrasonic sub-signal in the second ultrasonic signal is equal to a sum of the voltage amplitude of the ultrasonic sub-signal in the first ultrasonic signal and the voltage amplitude of the ultrasonic sub-signal in the third ultrasonic signal.

For example, the pulse sequence includes two ultrasonic signal groups, i.e., a first ultrasonic signal group and a second ultrasonic signal group. The first ultrasonic signal group includes a first ultrasonic signal, a second ultrasonic signal and a third ultrasonic signal; and the second ultrasonic signal group includes a fourth ultrasonic signal, a fifth ultrasonic signal and a sixth ultrasonic signal. Each ultrasonic signal includes two periods of ultrasonic sub-signals. The first ultrasonic signal has a voltage amplitude of V1, and includes an ultrasonic sub-signal f1 in a first period and an ultrasonic sub-signal f2 in a second period. The second ultrasonic signal has a voltage amplitude of V2, and includes an ultrasonic sub-signal f1 in the first period and an ultrasonic sub-signal f2 in the second period. The third ultrasonic signal has a voltage amplitude of V3, and includes an ultrasonic sub-signal f1 in the first period and an ultrasonic sub-signal f2 in the second period. The fourth ultrasonic signal has a voltage amplitude of V4, and includes an ultrasonic sub-signal f3 in the first period and an ultrasonic sub-signal f4 in the second period. The fifth ultrasonic signal has a voltage amplitude of V5, and includes an ultrasonic sub-signal f3 in the first period and an ultrasonic sub-signal f4 in the second period. The sixth ultrasonic signal has a voltage amplitude of V6, and includes an ultrasonic sub-signal f3 in the first period and an ultrasonic sub-signal f4 in the second period. When V1≠V2≠V3 and V4≠V5≠V6, the number of array elements of the transmitting aperture is the same when the ultrasonic signals are transmitted. Preferably, V2=2×V1=2×V3 and V5=2×V4=2×V6.

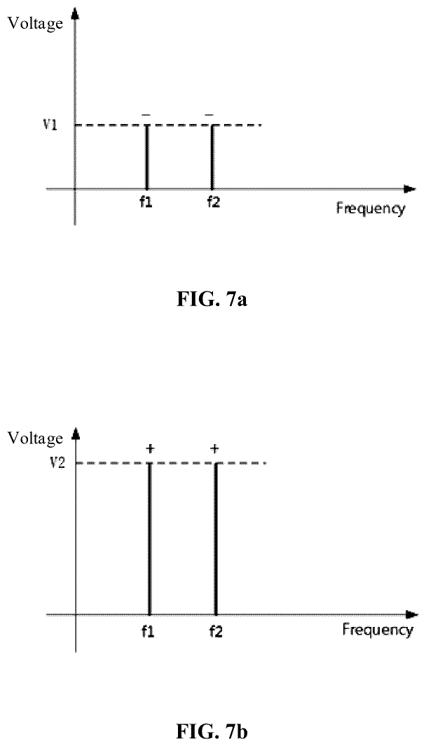

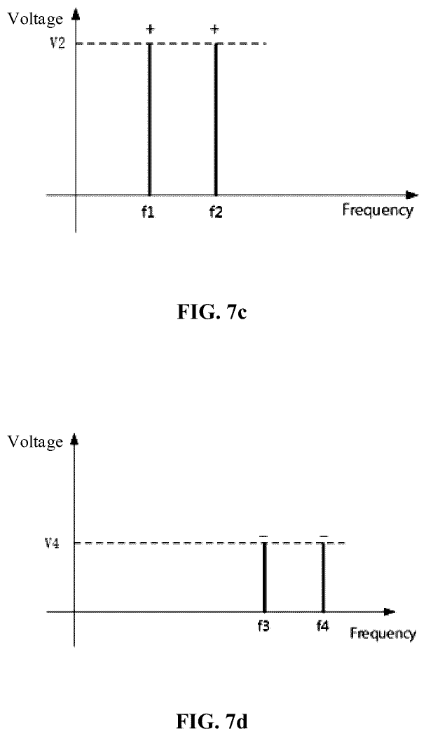

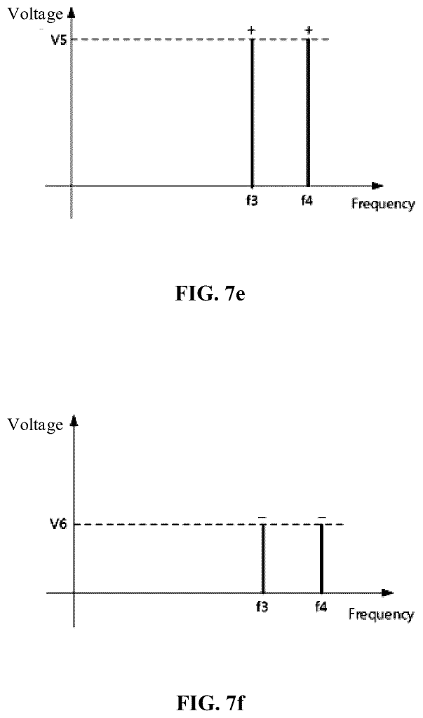

It should be noted that the first ultrasonic signal and the third ultrasonic signal are in phase, and the second ultrasonic signal and the first ultrasonic signal may be in phase or out of phase. Similarly, the fourth ultrasonic signal the sixth ultrasonic signal are in phase, and the fifth ultrasonic signal the fourth ultrasonic signal may be in phase or out of phase. This is not specifically limited here. For example, the first ultrasonic signal is as shown in FIG. 7a, the second ultrasonic signal is as shown in FIG. 7b, the third ultrasonic signal is as shown in FIG. 7c, the fourth ultrasonic signal is as shown in FIG. 7d, the fifth ultrasonic signal is as shown in FIG. 7e, and the sixth ultrasonic signal is as shown in FIG. 7f.

As another feasible implementation, the step S102 may include: determining the same voltage amplitude for the ultrasonic sub-signals in the corresponding period in the multiple ultrasonic signals of the same ultrasonic signal group; determining different numbers of array elements of the transmitting aperture for the ultrasonic sub-signals in the multiple ultrasonic signals of the same ultrasonic signal group; and transmitting the multiple ultrasonic signals in the multiple ultrasonic signal groups in the pulse sequence sequentially based on transmission frequencies, voltage amplitudes and the numbers of array elements of the transmitting aperture for different ultrasonic sub-signals in the multiple ultrasonic signals. That is, in the same ultrasonic signal group, voltage amplitudes of the multiple ultrasonic signals in the corresponding period are the same, and the numbers of array elements of the transmitting aperture for transmitting the multiple ultrasonic signals in the same ultrasonic signal are different. A transmitting energy may be adjusted by controlling the number of array elements of the transmitting aperture, which is equivalent to adjusting a voltage amplitude in the transmission. Hence, different ultrasonic signals in the same ultrasonic signal group received by the contrast agent microbubbles have different voltage amplitudes.

In the case where each ultrasonic signal group includes two ultrasonic signals, i.e., a first ultrasonic signal and a second ultrasonic signal, the determining different numbers of array elements of the transmitting aperture for the ultrasonic sub-signals in the multiple ultrasonic signals of the same ultrasonic signal group includes: determining a first number of array elements of the transmitting aperture for an ultrasonic sub-signal in the first ultrasonic signal, and determining a second number of array elements of the transmitting aperture for an ultrasonic sub-signal in the second ultrasonic signal, where the second number of array elements of the transmitting aperture is a preset multiple of the first number of array elements of the transmitting aperture. In a specific implementation, the number of array elements of the transmitting aperture for the ultrasonic sub-signal in the second ultrasonic signal is a preset multiple of the number of array elements of the transmitting aperture of an ultrasonic sub-signal in the first ultrasonic signal, and a voltage amplitude of the ultrasonic sub-signal in the first ultrasonic signal is the same as a voltage amplitude of the ultrasonic sub-signal in the second ultrasonic signal. In this case, it can be realized that voltage amplitudes of the first ultrasonic signal and the second ultrasonic signal received by the contrast agent microbubbles are different.

In an embodiment, the preset multiple may be a multiple more than 1, that is, the second number of array elements of the transmitting aperture is greater than the first number of array elements of the transmitting aperture. In addition, the preset multiple may be an integer multiple or a non-integer multiple.

It should be noted that, in order to facilitate subsequent processing of the echo signals, preferably, the second number of array elements of the transmitting aperture is twice the first number of array elements of the transmitting aperture. That is, the number of array elements of the transmitting aperture for the ultrasonic sub-signal in the second ultrasonic signal is twice the number of array elements of the transmitting aperture for the ultrasonic sub-signal in the first ultrasonic signal.

For example, the pulse sequence includes two ultrasonic signal groups, i.e., a first ultrasonic signal group and a second ultrasonic signal group. The first ultrasonic signal group includes a first ultrasonic signal and a second ultrasonic signal, and the second ultrasonic signal group includes a third ultrasonic signal and a fourth ultrasonic signal. Each ultrasonic signal includes two periods of ultrasonic sub-signals. The first ultrasonic signal has a voltage amplitude of V1, and includes an ultrasonic sub-signal f1 in a first period and an ultrasonic sub-signal f2 in a second period. The second ultrasonic signal has a voltage amplitude of V2, and includes an ultrasonic sub-signal f1 in the first period and an ultrasonic sub-signal f2 in the second period. The third ultrasonic signal has a voltage amplitude of V3, and includes an ultrasonic sub-signal f3 in the first period and an ultrasonic sub-signal f4 in the second period. The fourth ultrasonic signal has a voltage amplitude of V4, and includes an ultrasonic sub-signal f3 in the first period and an ultrasonic sub-signal f4 in the second period. When V1=V2 and V3=V4, the first ultrasonic signal and the third ultrasonic signal may be transmitted through odd apertures or even apertures, and the second ultrasonic signal and the fourth ultrasonic signal may be transmitted through full apertures.

In the case where each ultrasonic signal group includes three ultrasonic signals, i.e., a first ultrasonic signal, a second ultrasonic signal and a third ultrasonic signal, the determining different numbers of array elements of the transmitting aperture for the ultrasonic sub-signals in the multiple ultrasonic signals of the same ultrasonic signal group includes: determining the transmit aperture of the ultrasonic sub-signal in the first ultrasonic signal as one of an odd aperture and an even aperture, determining the transmit aperture of the ultrasonic sub-signal in the second ultrasonic signal as a full aperture, and determining the transmit aperture of the ultrasonic sub-signal in the third ultrasonic signal as the other one of the odd aperture and the even aperture. In a specific implementation, voltage amplitudes of different ultrasonic signals in the same ultrasonic signal group in the corresponding period are the same, the first ultrasonic signal may be transmitted through one of the odd aperture and the even aperture, the second ultrasonic signal may be transmitted through the full aperture, and the third ultrasonic signal may be transmitted through the other one of the odd aperture and the even aperture. In this way, it can be realized that voltage amplitudes of the first ultrasonic signal and the second ultrasonic signal received by the contrast agent microbubbles are different. In an embodiment, when the transmit aperture of the ultrasonic sub-signal in the first ultrasonic signal is determined as the odd aperture, the transmit aperture of the ultrasonic sub-signal in the third ultrasonic signal is the even aperture.

Furthermore, array elements at an odd position and array elements at an even position may be determined based on arrangement of the array elements. The transmitting aperture is determined as the odd aperture by determining the array elements at the odd position as the array elements of the transmitting aperture, that is, determining the number of array elements at odd position as the number of array elements of the transmitting aperture. The transmitting aperture is determined as the even aperture by determining the array elements at the even position as array elements of the transmitting aperture, that is, determining the number of array elements at the even position as the number of array elements of the transmitting aperture. The transmitting aperture is determined as the full aperture by determining all array elements as array elements of the transmitting aperture, that is, determining the total number of array elements as the number of array elements of the transmitting aperture.

For example, the pulse sequence includes two ultrasonic signal groups, i.e., a first ultrasonic signal group and a second ultrasonic signal group. The first ultrasonic signal group includes a first ultrasonic signal, a second ultrasonic signal and a third ultrasonic signal, and the second ultrasonic signal group includes a fourth ultrasonic signal, a fifth ultrasonic signal and a sixth ultrasonic signal. Each ultrasonic signal includes two periods of ultrasonic sub-signals. The first ultrasonic signal has a voltage amplitude of V1, and includes an ultrasonic sub-signal f1 in a first period and an ultrasonic sub-signal f2 in a second period. The second ultrasonic signal has a voltage amplitude of V2, and includes an ultrasonic sub-signal f1 in the first period and an ultrasonic sub-signal f2 in the second period. The third ultrasonic signal has a voltage amplitude of V3, and includes an ultrasonic sub-signal f1 in the first period and an ultrasonic sub-signal f2 in the second period. The fourth ultrasonic signal has a voltage amplitude of V4, and includes an ultrasonic sub-signal f3 in the first period and an ultrasonic sub-signal f4 in the second period. The fifth ultrasonic signal has a voltage amplitude of V5, and includes an ultrasonic sub-signal f3 in the first period and an ultrasonic sub-signal f4 in the second period. The sixth ultrasonic signal has a voltage amplitude of V6, and includes an ultrasonic sub-signal f3 in the first period and an ultrasonic sub-signal f4 in the second period. When V1=V2=V3 and V4=V5=V6, the numbers of array elements of the transmitting aperture are different when ultrasonic signals are transmitted. In an embodiment, the number of array elements of the transmitting aperture for transmitting the second ultrasonic signal is twice the number of array elements of the transmitting aperture for transmitting the first ultrasonic signal or the third ultrasonic signal. The number of array elements of the transmitting aperture for transmitting the fifth ultrasonic signal is twice the number of array elements of the transmitting aperture for transmitting the fourth ultrasonic signal or the sixth ultrasonic signal. That is, the number of array elements of the transmitting aperture for the second ultrasonic signal is a sum of the numbers of array elements of the transmitting aperture for the first ultrasonic signal and the third ultrasonic signal. The number of array elements of the transmitting aperture for the fifth ultrasonic signal is a sum of the numbers of array elements of the transmitting aperture for the fourth ultrasonic signal and the sixth ultrasonic signal. In a specific implementation, the first ultrasonic signal and the fourth ultrasonic signal may be transmitted through one of the odd aperture and the even aperture, the second ultrasonic signal and the fifth ultrasonic signal may be transmitted through the full aperture, and the third ultrasonic signal and the sixth ultrasonic signal may be transmitted through the other one of the odd aperture and the even aperture.

In an implementation, the transmitting the multiple ultrasonic signals in the multiple ultrasonic signal groups in the pulse sequence sequentially and receiving echo signals generated by contrast agent microbubbles in response to the transmitted ultrasonic signals includes: transmitting the multiple ultrasonic signals in the multiple ultrasonic signal groups in the pulse sequence sequentially, where in response the multiple ultrasonic signals, the contrast agent microbubbles generate fundamental signals corresponding to different first imaging frequencies, generate multi-harmonic signals corresponding to a second imaging frequency based on the fundamental signals corresponding to the first imaging frequencies, and generate a differential harmonic signal corresponding to a third imaging frequency based on the fundamental signals corresponding to the different first imaging frequencies, where the second imaging frequency is an integer multiple of the first imaging frequency, and the third imaging frequency is at least one of a difference between different first imaging frequencies or a sum of different first imaging frequencies; and receiving the fundamental signals corresponding to the first imaging frequencies, the multi-harmonic signal corresponding to the second imaging frequency, and the differential harmonic signal corresponding to the third imaging frequency. In an embodiment, a difference between different first imaging frequencies may be determined, a sum of different first imaging frequencies may be determined, and the difference and/or the sum may be determined as the third imaging frequency.

In a specific implementation, the ultrasonic probe transmits the pulse sequence, and the contrast agent microbubbles generate the echo signal. The echo signal may include fundamental signals and harmonic signals. The imaging frequencies of the fundamental signals (that is, the first imaging frequencies) are equal to the transmission frequencies. The harmonic signals include differential harmonic signals and multi-harmonic signals. The differential harmonic signal is a differential signal of fundamental signals. An imaging frequency of the differential harmonic signal (that is, the third imaging frequency) is a difference between different transmission frequencies and/or a sum of different transmission frequencies. The multi-harmonic signals may be understood as multiple superposition of the same fundamental signal. For example, the multi-harmonic signals may be a second-harmonic signal, or a third-harmonic signal. The imaging frequency of the multi-harmonic signals (that is, the second imaging frequency) is an integer multiple of a transmission frequency. Harmonic signals in third or higher order often exceed the effective bandwidth range of the ultrasonic probe, and therefore are generally not considered.

An example is provided here based on FIG. 4a to FIG. 4d. For the first ultrasonic signal group, a nonlinear fundamental signal of 0.5f and a nonlinear fundamental signal of f may be directly obtained. A differential harmonic signal of 0.5f and a differential harmonic signal of 1.5f may be obtained through a product-to-sum operation on the nonlinear fundamental signal of 0.5f and the nonlinear fundamental signal of f. In an embodiment, the differential harmonic signal of 0.5f may be obtained by calculating a difference between the nonlinear fundamental signal of 0.5f and the nonlinear fundamental signal of f; and the differential harmonic signal of 1.5f may be obtained by calculating a sum of the nonlinear fundamental signal of 0.5f and the nonlinear fundamental signal of f.

A second harmonic signal of 0.5f, that is, a harmonic signal of f, may be obtained, based on the nonlinear fundamental signal of 0.5f corresponding to the first ultrasonic signal and the nonlinear fundamental signal of 0.5f corresponding to the second ultrasonic signal. A second harmonic signal of f, that is, a harmonic signal of 2f, may be obtained, based on the nonlinear fundamental signal of f corresponding to the first ultrasonic signal and the nonlinear fundamental signal of f corresponding to the second ultrasonic signal. Therefore, contrast signals corresponding to the first ultrasonic signal group include the nonlinear fundamental signal of 0.5f, the nonlinear fundamental signal of f, the differential harmonic signal of 0.5f, the differential harmonic signal of 1.5f, the harmonic signal of f and the harmonic signal of 2f. Similarly, contrast signals corresponding to the second ultrasonic signal group include the nonlinear fundamental signal of 1.5f, the nonlinear fundamental signal of 2f, the differential harmonic signal of 0.5f, the differential harmonic signal of 3.5f, the harmonic signal of 3f, and the harmonic signal of 4f.

In S103, the echo signals are processed to obtain multiple contrast signals corresponding to different imaging frequencies, and contrast images are generated based on the contrast signals.

In the step S103, the echo signals of the multiple ultrasonic signals are processed to obtain multiple contrast signals corresponding to different imaging frequencies. The imaging frequency may be equal to the transmission frequency or an integer multiple of the transmission frequency. The contrast signals include fundamental signals and harmonic signals of multiple imaging frequencies, and the fundamental signals and the harmonic signals corresponding to multiple imaging frequencies enhance each other to obtain enhanced contrast signals. Signal processing is performed on the enhanced contrast signals to generate contrast images and then and store the contrast images.

As a feasible implementation, the processing the echo signals to obtain multiple contrast signals corresponding to different imaging frequencies includes: performing weighted processing on the echo signals of the multiple ultrasonic signals in each of the ultrasonic signal groups, based on a voltage amplitude relationship and a phase relationship among the multiple ultrasonic signals in each of the ultrasonic signal groups, to obtain the contrast signals corresponding to the respective ultrasonic signal groups; and demodulating the contrast signals corresponding to the respective ultrasonic signal groups by using demodulation circuits corresponding to different imaging frequencies, to obtain the contrast signals corresponding to the different imaging frequencies.

In a specific implementation, weighting coefficients of the echo signals are determined based on the voltage amplitude relationship among the multiple ultrasonic signals in each of the ultrasonic signal groups. In the example shown in FIG. 6a to FIG. 6d, when V2=Weight_Path1×V1, a weight corresponding to the first ultrasonic signal is Weight_Path1, and a weight corresponding to the second ultrasonic signal is 1. When V4=Weight_Path2×V3, a weight corresponding to the third ultrasonic signal is Weight_Path2, and a weight corresponding to the fourth ultrasonic signal is 1. In the example shown in FIG. 7a to FIG. 7f, when V1=V3=V2/Weight_Path1, a weight corresponding to the first ultrasonic signal and the third ultrasonic signal is 1, and a weight corresponding to the second ultrasonic signal is 2/Weight_Path1. When V4=V6=V5/Weight_Path2, a weight corresponding to the fourth ultrasonic signal and the sixth ultrasonic signal is 1, and a weight corresponding to the fifth ultrasonic signal is 2/Weight_Path2.

In a specific implementation, if different ultrasonic sub-signals in an ultrasonic signal have different voltage amplitudes, weights may be set for the different ultrasonic sub-signals in the ultrasonic signal based on a proportional relationship of the voltage amplitudes. The set weights of the ultrasonic sub-signals and the weights of the ultrasonic signal determined in the previous embodiment are superimposed and subjected to weighted processing to obtain the contrast signals corresponding to the ultrasonic signal group.

Further, an operational relationship of the echo signals is determined based on the phase relationship among the multiple ultrasonic signals in each of the ultrasonic signal groups, where the operational relationship is determined as an addition in response to the phase relationship being out of phase, and an operational relationship is determined as a subtraction in response to the phase relationship being in phase. That is, if the phase relationship among the multiple ultrasonic signals in the ultrasonic signal group is out of phase, then during the weighted processing, weighted summation is performed on the echo signals corresponding to the multiple ultrasonic signals in the ultrasonic signal group. If the phase relationship among the multiple ultrasonic signals in the ultrasonic signal group is in phase, then during the weighted processing, weighted subtraction is performed on the echo signals corresponding to the multiple ultrasonic signals in the ultrasonic signal group. The processing of performing addition for out of phase and performing subtraction for in phase can offset a fundamental signal of a tissue, and leave nonlinear fundamental signals. In addition, the harmonic signal, due to the square, is always enhanced regardless of addition or subtraction. That is, through such processing, irrelevant signals in the echo signal can be removed to ensure a reliability of the contrast image.

Based on the determined weights and operational relationship, weighted processing is performed on the echo signals of the multiple ultrasonic signals in each ultrasonic signal groups, to obtain the contrast signal corresponding to each ultrasonic signal group. Taking FIG. 6a to FIG. 6d as an example, in the case where the first ultrasonic signal and the second ultrasonic signal in the first ultrasonic signal group are out of phase, a weighted summation is performed on the first ultrasonic signal and the second ultrasonic signal in the process of calculating the contrast signal corresponding to the first ultrasonic signal group. In the case where the first ultrasonic signal and the second ultrasonic signal in the second ultrasonic signal group are out of phase, a weighted summation is performed on the first ultrasonic signal and the second ultrasonic signal in the process of calculating the contrast signal corresponding to the second ultrasonic signal group. The contrast signal Dataout1 corresponding to the first ultrasonic signal group and the contrast signal Dataout2 corresponding to the second ultrasonic signal group are:

$$Dataout 1 = Weight\_Path 1 \times Data\_ 1 stTX + Data\_ 2 ndTX ;$$

$$Dataout 2 = Weight\_Path 2 \times Data\_ 3 rdTX + Data\_ 4 thTX .$$

In the formula, Data_1stTX represents an echo signal of the first ultrasonic signal, Data_2ndTX represents an echo signal of the second ultrasonic signal, Data_3rdTX represents an echo signal of the third ultrasonic signal, and Data_4thTX represents an echo signal of the fourth ultrasonic signal.

Additionally, in the case where the first ultrasonic signal and the second ultrasonic signal in the first ultrasonic signal group are in phase, a weighted subtraction is performed on the first ultrasonic signal and the second ultrasonic signal in the process of calculating the contrast signal corresponding to the first ultrasonic signal group. In the case where the first ultrasonic signal and the second ultrasonic signal in the second ultrasonic signal group are in phase, a weighted subtraction is performed on the first ultrasonic signal and the second ultrasonic signal in the process of calculating the contrast signal corresponding to the second ultrasonic signal group. That is:

$$Dataout 1 = Weight\_Path 1 \times Data\_ 1 stTX - Data\_ 2 ndTX ;$$

$$Dataout 2 = Weight\_Path 2 \times Data\_ 3 rdTX - Data\_ 4 thTX .$$

In the case where the first ultrasonic signal and the second ultrasonic signal in the first ultrasonic signal group are out of phase, a weighted summation is performed on the first ultrasonic signal and the second ultrasonic signal in the process of calculating the contrast signal corresponding to the first ultrasonic signal group. In the case where the first ultrasonic signal and the second ultrasonic signal in the second ultrasonic signal group are in phase, a weighted subtraction is performed on the first ultrasonic signal and the second ultrasonic signal in the process of calculating the contrast signal corresponding to the second ultrasonic signal group. That is:

$$Dataout 1 = Weight\_Path 1 \times Data\_ 1 stTX + Data\_ 2 ndTX ;$$

$$Dataout 2 = Weight\_Path 2 \times Data\_ 3 rdTX - Data\_ 4 thTX .$$

In the case where the first ultrasonic signal and the second ultrasonic signal in the first ultrasonic signal group are in phase, a weighted subtraction is performed on the first ultrasonic signal and the second ultrasonic signal in the process of calculating the contrast signal corresponding to the first ultrasonic signal group. In the case where the first ultrasonic signal and the second ultrasonic signal in the second ultrasonic signal group are out of phase, a weighted summation is performed on the first ultrasonic signal and the second ultrasonic signal in the process of calculating the contrast signal corresponding to the first ultrasonic signal group. That is:

$$Dataout 1 = Weight\_Path 1 \times Data\_ 1 stTX - Data\_ 2 ndTX ;$$

$$Dataout 2 = Weight\_Path 2 \times Data\_ 3 rdTX + Data\_ 4 thTX .$$

For each ultrasonic signal group including three ultrasonic signals, in the case where the first ultrasonic signal is in phase with the third ultrasonic signal and is in phase or out of phase with the second ultrasonic signal, a summation (which may be a weighted summation) may be performed on the first ultrasonic signal and the third ultrasonic signal, and then weighted processing is performed on the second ultrasonic signal and the summation of the first ultrasonic signal and the third ultrasonic signal. Specifically, in the case where the first ultrasonic signal is in phase with the second ultrasonic signal, a weighted subtraction is performed on the second ultrasonic signal and the summation of the first ultrasonic signal and the third ultrasonic signal; and in the case where the first ultrasonic signal is out of phase with the second ultrasonic signal, a weighted summation is performed on the second ultrasonic signal and the summation of the first ultrasonic signal and the third ultrasonic signal.

Taking FIG. 7a to FIG. 7f as an example, in the case where the first ultrasonic signal is in phase with the third ultrasonic signal and is out of phase with the second ultrasonic signal in the first ultrasonic signal group, a weighted summation is performed on the third ultrasonic signal and a sum of the first ultrasonic signal and the second ultrasonic signal in the process of calculating the contrast signal corresponding to the first ultrasonic signal group. In the case where the fourth ultrasonic signal is in phase with the sixth ultrasonic signal and is out of phase with the fifth ultrasonic signal in the second ultrasonic signal group, a weighted summation is performed on the sixth ultrasonic signal and a sum of the fourth ultrasonic signal and the fifth ultrasonic signal in the process of calculating the contrast signal corresponding to the second ultrasonic signal group. A contrast signal Dataout1 corresponding to the first ultrasonic signal group and a contrast signal Dataout2 corresponding to the second ultrasonic signal group are:

$$Dataout 1 = Data\_ 1 stTX + 2 / Weight\_Path 1 \times Data\_ 2 ndTX + Data\_ 3 rdTX ;$$

$$Dataout 2 = Data\_ 4 thTX + 2 / Weight\_Path 2 \times Data\_ 5 thTX + Data\_ 6 thTX .$$

In the formula, Data_1stTX represents an echo signal of the first ultrasonic signal, Data_2ndTX represents an echo signal of the second ultrasonic signal, Data_3rdTX represents an echo signal of the third ultrasonic signal, Data_4thTX represents an echo signal of the fourth ultrasonic signal, Data_5thTX represents an echo signal of the fifth ultrasonic signal, and Data_6thTX represents an echo signal of the sixth ultrasonic signal.

Additionally, in the case where the first ultrasonic signal, the second ultrasonic signal and the third ultrasonic signal are in phase in the first ultrasonic signal group, a weighted subtraction is performed on the third ultrasonic signal and a sum of the first ultrasonic signal and the second ultrasonic signal in the process of calculating the contrast signal corresponding to the first ultrasonic signal group. In the case where the fourth ultrasonic signal is in phase with the sixth ultrasonic signal and is out of phase with the fifth ultrasonic signal in the second ultrasonic signal group, a weighted summation is performed on the sixth ultrasonic signal and a sum of the fourth ultrasonic signal and the fifth ultrasonic signal in the process of calculating the contrast signal corresponding to the second ultrasonic signal group. A contrast signal Dataout1 corresponding to the first ultrasonic signal group and a contrast signal Dataout2 corresponding to the second ultrasonic signal group are:

$$Dataout 1 = Data\_ 1 stTX - 2 / Weight\_Path 1 \times Data\_ 2 ndTX + Data\_ 3 rdTX ;$$

$$Dataout 2 = Data\_ 4 thTX + 2 / Weight\_Path 2 \times Data\_ 5 thTX + Data\_ 6 thTX .$$

Further, it is necessary to demodulate the contrast signals corresponding to each of the ultrasonic signal groups by using demodulation circuits corresponding to different imaging frequencies, to obtain the contrast signals corresponding to different imaging frequencies. Taking FIG. 4a to FIG. 4d as an example, the contrast signals corresponding to the first ultrasonic signal group are demodulated by using demodulation circuits corresponding to 0.5f, f, 1.5f, and 2f, to obtain a first contrast signal of 0.5f (including a nonlinear fundamental signal of 0.5f and a differential harmonic signal of 0.5f), a second contrast signal of f (including a nonlinear fundamental signal of f and a harmonic signal of f), a third contrast signal of 1.5f (including a differential harmonic signal of 1.5f and a third harmonic signal of 1.5f), and a fourth contrast signal of 2f (including a harmonic signal of 2f). Furthermore, 3f, 3.5f, and 4f exceed the effective bandwidth range of the ultrasonic probe. Hence, the contrast signals corresponding to the second ultrasonic signal group are demodulated by using demodulation circuits corresponding to 0.5f, 1.5f, and 2f, to obtain a fifth contrast signal of 0.5f (including a differential harmonic signal of 0.5f), a sixth contrast signal of 1.5f (including a nonlinear fundamental signal of 1.5f), and a seventh contrast signal of 2f (including a nonlinear fundamental signal of 2f). Thus, 4 demodulation circuits are used to demodulate the contrast signals. After subsequent signal processing, seven contrast images corresponding to seven contrast signals are generated, as shown in Table 1.

TABLE 1

| | Contrast | | | | | Demodulation |
|---|---|---|---|---|---|---|
| Pulse sequence | signal | Frequency | | Frequency component | Image | circuit |
| First ultrasonic | First | 0.5 | f | nonlinear fundamental | First | 1st |
| signal group | contrast | | | wave of 0.5 f + differential | contrast | demodulation |
| | signal | | | harmonic wave of 0.5 f | image | circuit |
| First ultrasonic | Second | f | | nonlinear fundamental | Second | 2nd |
| signal group | contrast | | | wave of f + harmonic | contrast | demodulation |
| | signal | | | wave of f | image | circuit |
| First ultrasonic | Third | 1.5 | f | differential harmonic | Third | 3rd |
| signal group | contrast | | | wave of 1.5 f + third | contrast | demodulation |
| | signal | | | harmonic wave of 1.5 f | image | circuit |
| First ultrasonic | Fourth | 2 | f | harmonic wave of 2 f | Fourth | 4th |
| signal group | contrast | | | | contrast | demodulation |
| | signal | | | | image | circuit |
| Second | Fifth | 0.5 | f | differential harmonic | Fifth | 1st |
| ultrasonic | contrast | | | wave of 0.5 f | contrast | demodulation |
| signal group | signal | | | | image | circuit |
| Second | Sixth | 1.5 | f | nonlinear fundamental | Sixth | 3rd |
| ultrasonic | contrast | | | wave of 1.5 f | contrast | demodulation |
| signal group | signal | | | | image | circuit |
| Second | Seventh | 2 | f | nonlinear fundamental | Seventh | 4th |
| ultrasonic | contrast | | | wave of 2 f | contrast | demodulation |
| signal group | signal | | | | image | circuit |

Taking FIG. 5a to FIG. 5d as an example, if f=f, contrast signals corresponding to the first ultrasonic signal group include a nonlinear fundamental signal of f and a harmonic signal of 2f, and contrast signals corresponding to the second ultrasonic signal group include a nonlinear fundamental signal of 1.5f, a nonlinear fundamental signal of 2f, a differential harmonic signal of 0.5f, a differential harmonic signal of 3.5f, a harmonic signal of 3f, and a harmonic signal of 4f. Frequencies 3f, 3.5f, and 4f exceed the effective bandwidth range of the ultrasonic probe. Hence, the contrast signals corresponding to the first ultrasonic signal group are demodulated by using demodulation circuits corresponding to f and 2f, to obtain a first contrast signal of f (including the nonlinear fundamental signal of f) and a second contrast signal of 2f (including the harmonic signal of 2f). The contrast signals corresponding to the second ultrasonic signal group is demodulated by using demodulation circuits corresponding to 0.5f, 1.5f, and 2f, to obtain a third contrast signal of 0.5f (including a differential harmonic signal of 0.5f), a fourth contrast signal of 1.5f (including a nonlinear fundamental signal of 1.5f), and a fifth contrast signal of 2f (including a nonlinear fundamental signal of 2f). Thus, 4 demodulation circuits are used to demodulate the contrast signals. After subsequent signal processing, five contrast images corresponding to five contrast signals are generated, as shown in Table 2.

TABLE 2

| Pulse | Contrast | | | | | Demodulation |
|---|---|---|---|---|---|---|
| sequence | signal | Frequency | | Frequency component | Image | circuit |
| First | First contrast | f | | nonlinear fundamental | First contrast | 2nd |
| ultrasonic | signal | | | wave of f | image | demodulation |
| signal group | | | | | | circuit |
| First | Second | 2 | f | harmonic wave of 2 f | Second | 4th |
| ultrasonic | contrast | | | | contrast | demodulation |
| signal group | signal | | | | image | circuit |
| Second | Third | 0.5 | f | differential harmonic | Third | 1st |
| ultrasonic | contrast | | | wave of 0.5 f | contrast | demodulation |
| signal group | signal | | | | image | circuit |
| Second | Fourth | 1.5 | f | nonlinear fundamental | Fourth | 3rd |
| ultrasonic | contrast | | | wave of 1.5 f | contrast | demodulation |
| signal group | signal | | | | image | circuit |
| Second | Fifth contrast | 2 | f | nonlinear fundamental | Fifth contrast | 4th |
| ultrasonic | signal | | | wave of 2 f | image | demodulation |
| signal group | | | | | | circuit |

If f′ is slightly greater than f, contrast signals corresponding to the first ultrasonic signal group include a nonlinear fundamental signal of f, a nonlinear fundamental signal of f′, a differential harmonic signal of f′−f, a differential harmonic signal of f′+f, a harmonic signal of 2f and a harmonic signal of 2f′. Contrast signals corresponding to the second ultrasonic signal group include a nonlinear fundamental signal of 1.5f, a nonlinear fundamental signal of 2f, a differential harmonic signal of 0.5f, a differential harmonic signal of 3.5f, a harmonic signal of 3f, and a harmonic signal of 4f. Frequencies f′−f, 3f, 3.5f, and 4f exceed the effective bandwidth range of the ultrasonic probe, and f′+f is close to 2f. Hence, the contrast signal corresponding to the first ultrasonic signal group is demodulated by using demodulation circuits corresponding to f, 2f, f′, and 2f′, to obtain a first contrast signal of f (including a nonlinear fundamental signal of f), a second contrast signal of 2f (including a harmonic signal of 2f), a third contrast signal of f′ (including a nonlinear fundamental signal of f′), a fourth contrast signal of 2f′ (including a harmonic signal of 2f′). The contrast signal corresponding to the second ultrasonic signal group is demodulated by using demodulation circuits corresponding to 0.5f, 1.5f, and 2f, to obtain a fifth contrast signal of 0.5f (including a differential harmonic signal of 0.5f), a sixth contrast signal of 1.5f (including a nonlinear fundamental signal of 1.5f), and a seventh contrast signal of 2f (including a nonlinear fundamental signal of 2f). Thus, 6 demodulation circuits are used to demodulate the contrast signal. After subsequent signal processing, seven contrast images corresponding to seven contrast signals are generated, as shown in Table 3.

TABLE 3

| Pulse | | | | | | Demodulation |
|---|---|---|---|---|---|---|
| sequence | Contrast signal | Frequency | | Frequency component | Image | circuit |
| First | First contrast | f | | nonlinear | First | 2nd |
| ultrasonic | signal | | | fundamental wave of | contrast | demodulation |
| signal group | | | | f | image | circuit |
| First | Second | 2 | f | harmonic wave of 2 f | Second | 4th |
| ultrasonic | contrast signal | | | | contrast | demodulation |
| signal group | | | | | image | circuit |
| First | Third contrast | f′ | | nonlinear | Third | 5th |
| ultrasonic | signal | | | fundamental wave of | contrast | demodulation |
| signal group | | | | f′ | image | circuit |
| First | Fourth contrast | 2 | f′ | harmonic wave of 2 f′ | Fourth | 6th |
| ultrasonic | signal | | | | contrast | demodulation |
| signal group | | | | | image | circuit |
| Second | Fifth contrast | 0.5 | f | differential harmonic | Fifth | 1st |
| ultrasonic | signal | | | wave of 0.5 f | contrast | demodulation |
| signal group | | | | | image | circuit |
| Second | Sixth contrast | 1.5 | f | nonlinear | Sixth | 3rd |
| ultrasonic | signal | | | fundamental wave of | contrast | demodulation |
| signal group | | | | 1.5 f | image | circuit |
| Second | Seventh | 2 | f | nonlinear | Seventh | 4th |
| ultrasonic | contrast signal | | | fundamental wave of | contrast | demodulation |
| signal group | | | | 2 f | image | circuit |

In S104, the contrast images are fused to obtain a target contrast image.

In this step, multiple contrast images are fused to obtain the target contrast image. As a feasible implementation, the step may include: determining weighting coefficients of contrast images corresponding to the different imaging modes, and performing weighted fusion on the multiple contrast images based on the weighting coefficients of the contrast images, to obtain target contrast images corresponding to different imaging modes. In a specific implementation, the weighted fusion is performed on the contrast images based on the weighting coefficients of the contrast images, to obtain an outputted target contrast image. A formula for the fusion is:

$$ContrastImage = {\sum }_{i} coefi \times Sub\_imagei .$$

In the equation, Sub_imagei represents the i-th contrast image, coefi represents a weighting coefficient of the i-th contrast image, and ContrastImage represents a target contrast image.

It should be noted that the contrast images corresponding to the different imaging modes have different weighting coefficients, and the weighting coefficients are in a range of [0,1]. The weighted fusion is performed on multiple contrast images based on the weighting coefficients of contrast images corresponding to the different imaging modes, to obtain target contrast images corresponding to different imaging modes. The imaging modes may include a general mode, a penetration mode, a resolution mode, and the like. In this way, requirements of penetration, sensitivity and resolution are all met.

For the general mode, the contrast images having medium frequency component may be adopted for fusion. Taking Table 1 as an example, the second contrast image and the sixth contrast image may be fused, that is, the contrast image of f and the contrast image of 1.5f may be fused. Alternatively, the second contrast image, the fourth contrast image, and the seventh contrast image may be fused, that is, the contrast image of f and the contrast image of 2f are fused, Alternatively, the second contrast image, the fourth contrast image, the sixth contrast image, and the seventh contrast image may be fused, that is, the contrast image of f, the contrast image of 1.5f, and the contrast image of 2f are fused. Apparently, there are other fusion methods, which are not specifically limited in this embodiment.

In the penetration mode, penetration and sensitivity of a contrast image is required to be high. Therefore, contrast images having low frequency component may be adopted for fusion. That is, the weighting coefficient of the contrast image corresponding to the penetration mode is negatively correlated with the imaging frequency corresponding to the contrast image. Taking Table 1 as an example, the first contrast image, i.e., the contrast image of 0.5f, may be determined as a target contrast image. Alternatively, the second contrast image, i.e., the contrast image of f, may be determined as the target contrast image. Alternatively, the first contrast image and the second contrast image, i.e., the contrast image of 0.5f and the contrast image of f, may be fused. Apparently, there are other fusion methods, which are not specifically limited in this embodiment.

In the resolution mode, resolution of a contrast image is required to be high. Therefore, contrast images having high frequency component may be adopted for fusion. That is, the weighting coefficient of the contrast image corresponding to the resolution mode is positively correlated with the imaging frequency corresponding to the contrast image. Taking Table 1 as an example, the fourth contrast image, the sixth contrast image and the seventh contrast image may be fused. That is, the contrast image of 1.5f and the contrast images of 2f are fused. Alternatively, the fourth contrast image and the seventh contrast image may be fused. That is, the contrast images of 2f are fused. Apparently, other fusion methods are possible, which is not specifically limited in this embodiment.

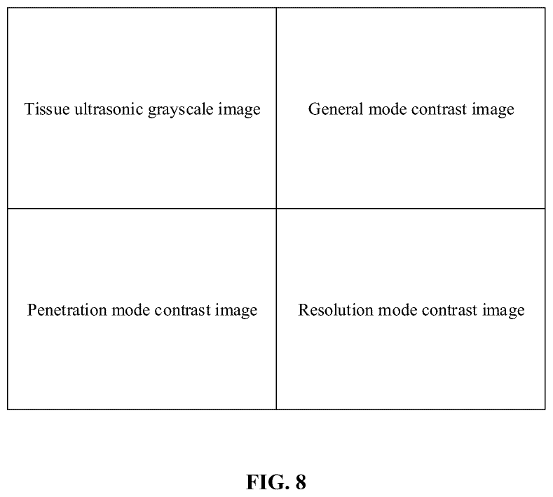

It can be understood that a user may select target contrast signals corresponding to one or more imaging modes for display. FIG. 8 shows a schematic display of an ultrasonic contrast image, in which a tissue ultrasonic grayscale image and target ultrasonic images in a general mode, a penetration mode and a resolution mode are displayed.

Alternatively, a user may adjust an arrangement order of the images by dragging the target ultrasonic image. When the user does not satisfy the adjustment result, an operation of one-click reset or rollback at least one step may be performed through a function key provided on the interface.

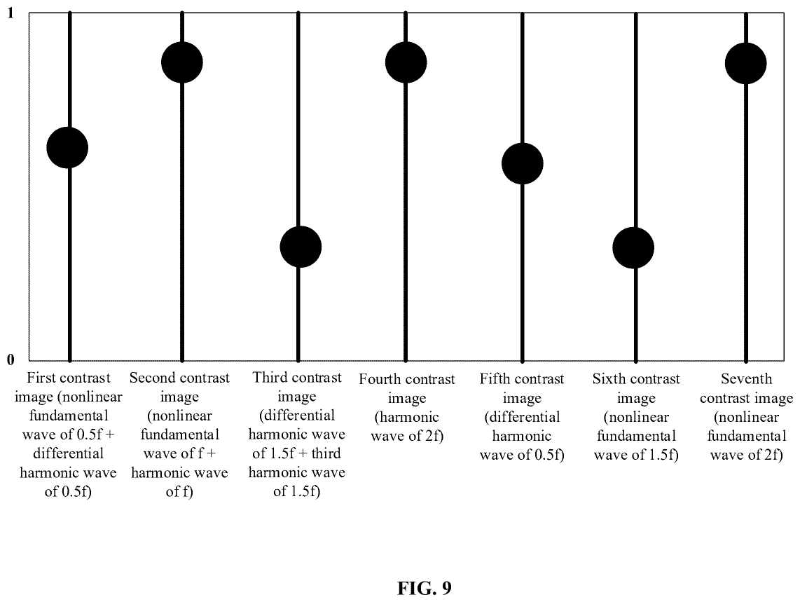

Furthermore, the contrast images corresponding to the contrast signals are generated and stored. Thus, a user may be allowed to manually adjust the weighting coefficients of the contrast images according to an embodiment. As a feasible implementation, the determining weighting coefficients of contrast images corresponding to the different imaging modes includes: displaying, for one target imaging mode, an adjustment window corresponding to the target contrast image, where the adjustment window includes adjustment areas for the contrast images; and receiving an adjustment instruction on at least one of the adjustment areas and adjusting the weighting coefficients of the contrast image corresponding to the at least one of the adjustment areas based on the adjustment instruction, to obtain weighting coefficients of contrast images in the target imaging mode. In a specific implementation, a user may click on a displayed target contrast image to display the adjustment window (as shown in FIG. 9) for the target contrast image. The user may drag a black dot corresponding to any one of contrast image up and down in the adjustment areas for the contrast images, to adjust to weighting coefficient to meet the needs of different scenarios. An adjustment rod (vertical line) and a black dot on the adjustment rod constitute an adjustment control for a contrast image. In the embodiment, personalized setting of the weighting coefficient is realized by adjusting the adjustment control, so that personalized adjustment of image fusion can be realized to meet the needs of different scenarios.

Alternatively, the ultrasonic device may include a display screen and a touch screen. The display screen is configured to output and display an ultrasonic image, and the like. The touch screen is configured to receive an input signal, such as key information inputted by a user through a virtual keyboard. Further, the ultrasonic host may be communicatively connected to the display screen and/or the touch screen, receive an adjustment instruction inputted from the display screen and/or the touch screen, obtain weighting coefficients of contrast images in a target imaging mode, and then realize weighted fusion of different contrast images by the ultrasonic host or the master computer.

The adjustment window may be displayed on the display screen and the touch screen, and the black dot corresponding to each contrast image may be slid by touch to adjust the weight of the contrast image. For an ultrasonic device without a touch screen, the adjustment window may be displayed on the display screen as a pop-up window. In this case, a knob or lever configured on the ultrasonic device may be operated to slide the black dot corresponding to the contrast image in the adjustment window, thereby adjusting the weight of the contrast image.

In an embodiment, different areas on the display screen of the ultrasonic device may serve as display windows for contrast images in different imaging modes. When a certain display window is selected, a target imaging mode corresponding to the selected window is determined to be selected, and an adjustment window for target contrast image corresponding to the target imaging mode is displayed. For an ultrasonic device with a touch screen, the adjustment window may be displayed directly on the touch screen. For an ultrasonic device without a touch screen, the adjustment window may pop up in a main display interface of the contrast image. Weighting coefficients of contrast images are determined based on the adjustment instruction on the adjustment window, and then the weighted fusion is performed to obtain the target contrast image.

With the method for ultrasonic contrast imaging provided by the present disclosure, the ultrasonic probe can obtain multiple contrast signals corresponding to different imaging frequencies through one-time transmission of a complete pulse sequence. The contrast signals include fundamental signals and harmonic signals of different imaging frequencies. A fundamental signal and a harmonic signal of one imaging frequency enhance each other, to obtain an enhanced contrast signal. The contrast image generated based on the enhanced contrast signal can meet requirements of penetration, sensitivity and resolution.

Hereinafter described is an apparatus for ultrasonic contrast imaging according to an embodiment of the present disclosure. The detail of the apparatus described below may refer to the method described above.

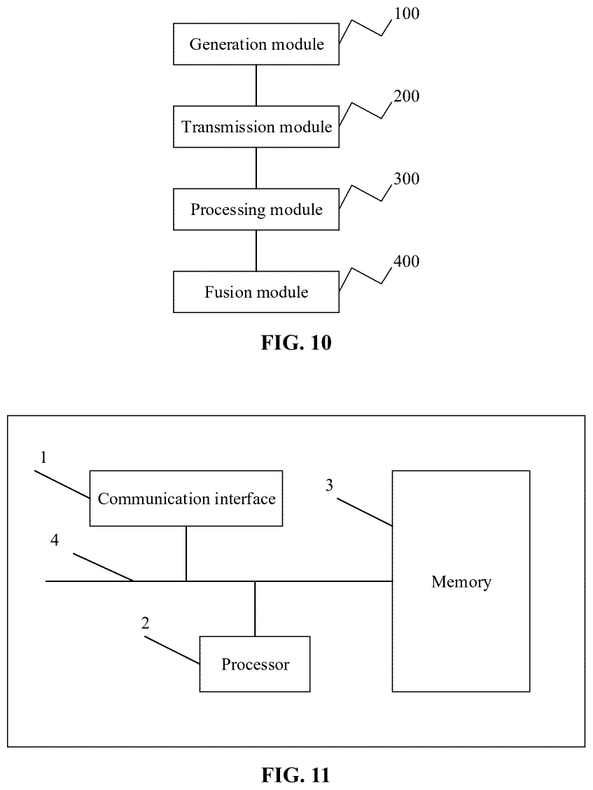

Reference is made to FIG. 10, which is a structural diagram of an apparatus for ultrasonic contrast imaging according to an exemplary embodiment. As shown in FIG. 10, the apparatus includes a generation module 100, a transmission module 200, a processing module 300, and a fusion module 400.

The generation module 100 is configured to determine multiple transmission frequencies within an effective bandwidth range of an ultrasonic probe, and generate a pulse sequence including multiple ultrasonic signal groups based on the transmission frequencies. Each of the ultrasonic signal groups includes multiple ultrasonic signals, and each of the ultrasonic signals includes at least two periods of ultrasonic sub-signals having different transmission frequencies. The multiple ultrasonic signals in the same ultrasonic signal group have the same transmission frequency in a corresponding period, and the multiple ultrasonic signals in different ultrasonic signal groups have different transmission frequencies in the corresponding period.

The transmission module 200 is configured to transmit the multiple ultrasonic signals in the multiple ultrasonic signal groups in the pulse sequence sequentially, and receive echo signals generated by contrast agent microbubbles in response to the transmitted ultrasonic signals. The echo signals include fundamental signals and harmonic signals corresponding to different imaging frequencies.

The processing module 300 is configured to process the echo signals to obtain multiple contrast signals corresponding to the different imaging frequencies, and generate contrast images based on the contrast signals.

The fusion module 400 is configured to fuse the contrast images to obtain a target contrast image.

With the apparatus for ultrasonic contrast imaging provided by the present disclosure, the ultrasonic probe can obtain multiple contrast signals corresponding to different imaging frequencies through one-time transmission of a complete pulse sequence. The contrast signals include fundamental signals and harmonic signals of different imaging frequencies. A fundamental signal and a harmonic signal of one imaging frequency enhance each other, so that an enhanced contrast signal is obtained. The contrast image generated based on the enhanced contrast signal can meet requirements of penetration, sensitivity and resolution.

Based on the above embodiment, as a preferred implementation, the generation module 100 is specifically configured to: determine a reference transmission frequency and a target coefficient, determine a product of the reference transmission frequency and the target coefficient as a first transmission frequency, determine twice the first transmission frequency as a second transmission frequency, determine three times the first transmission frequency as a third transmission frequency, and determine four times the first transmission frequency as a fourth transmission frequency, where the first transmission frequency, the second transmission frequency, the third transmission frequency and the fourth transmission frequency are all within the effective bandwidth range of the ultrasonic probe; and generate the pulse sequence including a first ultrasonic signal group and a second ultrasonic signal group, where the first ultrasonic signal group is generated based on the first transmission frequency and the second transmission frequency, and the second ultrasonic signal group is generated based on the third transmission frequency and the fourth transmission frequency.

Based on the above embodiment, as a preferred implementation, the generation module 100 is specifically configured to: determine a reference transmission frequency and a target coefficient, determine a product of the reference transmission frequency and the target coefficient as a first transmission frequency, determine twice the first transmission frequency as a second transmission frequency, determine three times the first transmission frequency as a third transmission frequency, determine four times the first transmission frequency as a fourth transmission frequency, and determine a transmission frequency adjacent to the second transmission as a fifth transmission frequency, where the second transmission frequency, the third transmission frequency, the fourth transmission frequency and the fifth transmission frequency are all within the effective bandwidth range of the ultrasonic probe; and generate the pulse sequence including a first ultrasonic signal group and a second ultrasonic signal group, where the first ultrasonic signal group is generated based on the fifth transmission frequency and the second transmission frequency, and the second ultrasonic signal group is generated based on the third transmission frequency and the fourth transmission frequency.

Based on the above embodiment, as a preferred implementation, the transmission module 200 includes a second determination unit, a third determination unit, and a first transmission unit.

The second determination unit is configured to determine different voltage amplitudes for ultrasonic sub-signals in the corresponding period in the multiple ultrasonic signals of the same ultrasonic signal group.

The third determination unit is configured to determine a same number of array elements of the transmitting aperture for ultrasonic sub-signals in the multiple ultrasonic signals of the same ultrasonic signal group.

The first transmission unit is configured to transmitting the multiple ultrasonic signals in the multiple ultrasonic signal groups in the pulse sequence sequentially based on transmission frequencies, voltage amplitudes and transmitting apertures for different ultrasonic sub-signals in the multiple ultrasonic signals.

Based on the above embodiment, as a preferred implementation, each of the ultrasonic signal groups includes a first ultrasonic signal and a second ultrasonic signal, and the second determination unit is specifically configured to: determine a first voltage amplitude for an ultrasonic sub-signal in the first ultrasonic signal, and determine a second voltage amplitude for an ultrasonic sub-signal in the second ultrasonic signal, where the second voltage amplitude is a preset multiple of the first voltage amplitude.

Based on the above embodiment, as a preferred implementation, the second voltage amplitude is twice the first voltage amplitude.

Based on the above embodiment, as a preferred implementation, each of the ultrasonic signal groups includes a first ultrasonic signal, a second ultrasonic signal and a third ultrasonic signal, and the second determination unit is specifically configured to: determine a third voltage amplitude for an ultrasonic sub-signal in the first ultrasonic signal and an ultrasonic sub-signal in the third ultrasonic signal, and determine a fourth voltage amplitude for an ultrasonic sub-signal in the second ultrasonic signal, where the fourth voltage amplitude is a preset multiple of the third voltage amplitude.

Based on the above embodiment, as a preferred implementation, the fourth voltage amplitude is twice the third voltage amplitude.

Based on the above embodiment, as a preferred implementation, the transmission module 200 includes a fourth determination unit, a fifth determination unit, and a second transmission unit.

The fourth determination unit is configured to determine the same voltage amplitude for the ultrasonic sub-signals in the corresponding period in the multiple ultrasonic signals of the same ultrasonic signal group.

The fifth determination unit is configured to determine different numbers of array elements of the transmitting aperture for the ultrasonic sub-signals in the multiple ultrasonic signals of the same ultrasonic signal group.

The second transmission unit is configured to transmitting the multiple ultrasonic signals in the multiple ultrasonic signal groups in the pulse sequence sequentially, based on transmission frequencies, voltage amplitudes and the transmitting aperture for different ultrasonic sub-signals in the multiple ultrasonic signals.

Based on the above embodiment, as a preferred implementation, each of the ultrasonic signal groups includes a first ultrasonic signal, a second ultrasonic signal and a third ultrasonic signal, and the fifth determination unit is specifically configured to: determine a first number of array elements of the transmitting aperture for an ultrasonic sub-signal in the first ultrasonic signal, and determine a second number of array elements of the transmitting aperture for an ultrasonic sub-signal in the second ultrasonic signal, where the second number of array elements of the transmitting aperture is a preset multiple of the first number of array elements of the transmitting aperture.

Based on the above embodiment, as a preferred implementation, the second number of array elements of the transmitting aperture is twice the first number of array elements of the transmitting aperture.

Based on the above embodiment, as a preferred implementation, each of the ultrasonic signal groups includes a first ultrasonic signal, a second ultrasonic signal and a third ultrasonic signal, and the fifth determination unit is specifically configured to: determine the transmitting aperture for the ultrasonic sub-signal in the first ultrasonic signal as one of an odd aperture and an even aperture, determine that the transmitting aperture for the ultrasonic sub-signal in the second ultrasonic signal as a full aperture, and determine the transmitting aperture for the ultrasonic sub-signal in the third ultrasonic signal as the other one of the odd aperture and the even aperture.

Based on the above embodiment, as a preferred implementation, the processing module 300 includes a processing unit, a demodulation unit, and a generation unit.

The processing unit is configured to perform weighted processing on echo signals of the multiple ultrasonic signals in each of the ultrasonic signal groups, based on a voltage amplitude relationship and a phase relationship among the multiple ultrasonic signals in each of the ultrasonic signal groups, to obtain the contrast signals corresponding to the respective ultrasonic signal groups.

The demodulation unit is configured to demodulate the contrast signals corresponding to each of the ultrasonic signal groups by using demodulation circuits corresponding to different imaging frequencies, to obtain the contrast signals corresponding to the different imaging frequencies.

The generation unit is configured to generate contrast images based on the contrast signals.

Based on the above embodiment, as a preferred implementation, the processing unit is specifically configured to: determine weighting coefficients of the echo signals based on the voltage amplitude relationship among the multiple ultrasonic signals in each of the ultrasonic signal groups; determine an operational relationship of the echo signals, based on the phase relationship among the multiple ultrasonic signals in each of the ultrasonic signal groups, where the operational relationship is determined as a summation in response to the phase relationship being out of phase, and the operational relationship is determined as a subtraction in response to the phase relationship being in phase; and perform weighted processing on the echo signals of the multiple ultrasonic signals in each of the ultrasonic signal groups based on the weighting coefficients and the operational relationship, to obtain the contrast signals corresponding to each of the ultrasonic signal groups.

Based on the above embodiment, as a preferred implementation, the transmission module 200 is specifically configured to: transmit the multiple ultrasonic signals in the multiple ultrasonic signal groups in the pulse sequence sequentially, where in response the multiple ultrasonic signals, the contrast agent microbubbles generate fundamental signals corresponding to different first imaging frequencies, generate a multi-harmonic signal corresponding to a second imaging frequency based on the fundamental signals corresponding to the first imaging frequencies, and generate a differential harmonic signal corresponding to a third imaging frequency based on the fundamental signals corresponding to the different first imaging frequencies, where the second imaging frequency is an integer multiple of the first imaging frequency, and the third imaging frequency is at least one of a difference between first imaging frequencies or a sum of different first imaging frequencies; and receive the fundamental signals corresponding to the first imaging frequencies, the multi-harmonic signal corresponding to the second imaging frequency, and the differential harmonic signal corresponding to the third imaging frequency.

Based on the above embodiment, as a preferred implementation, the fusion module 400 includes a first determination unit and a fusion unit.

The first determination unit is configured to determine weighting coefficients of contrast images corresponding to different imaging modes.

The fusion unit is configured to perform weighted fusion on the contrast images based on the weighting coefficients of the contrast images, to obtain target contrast images corresponding to different imaging modes.

Based on the above embodiment, as a preferred implementation, the imaging modes include a penetration mode and/or a resolution mode, the weighting coefficient of the contrast image corresponding to the penetration mode is negatively correlated with an imaging frequency corresponding to the contrast image, and the weighting coefficient of the contrast image corresponding to the resolution mode is positively correlated with the imaging frequency corresponding to the contrast image.

Based on the above embodiment, as a preferred implementation, the first determination unit is specifically configured to: display, for one target imaging mode, an adjustment window corresponding to the target contrast image, where the adjustment window includes adjustment areas for the contrast images; and receive an adjustment instruction on at least one of the adjustment areas and adjust the weighting coefficient of the contrast image corresponding to the at least one of the adjustment areas based on the adjustment instruction, to obtain the weighting coefficients of the contrast images in the target imaging mode.

Specific operations of modules in the apparatus in the above embodiments are described in detail in the embodiments related to the method, which will not be described in detail here.

Based on a hardware implementation of the above program modules, and to implement the method of the embodiments in the present disclosure, an ultrasonic imaging system is further provided in an embodiment of the present disclosure. The ultrasonic imaging system includes an ultrasonic host, an ultrasonic probe, and a master computer. The ultrasonic host is communicatively connected to the ultrasonic probe and the master computer.

The ultrasonic host is configured to perform the method for ultrasonic contrast imaging according to any of the above embodiments.

Based on a hardware implementation of the above program modules, and to implement the method of the embodiments in the present disclosure, an ultrasonic device is further provided in an embodiment of the present disclosure. FIG. 11 is a structural diagram of an ultrasonic device according to an exemplary embodiment. As shown in FIG. 11, the ultrasonic device includes a communication interface 1, a processor 2 and a memory 3.

The communication interface 1 is capable of performing information interaction with other devices such as a network device.

The processor 2 is connected to the communication interface 1 to perform information interaction with other devices, and is configured to execute the method for ultrasonic contrast imaging provided in one or more of the above technical solutions when executing a computer program. The computer program is stored in the memory 3.

Apparently, in practical applications, various components in the ultrasonic device are coupled together through a bus system 4. It can be understood that the bus system 4 is configured to realize connection and communication among the components. In addition to the data bus, the bus system 4 further includes a power bus, a control bus and a status signal bus. For the sake of clarity, the various buses are labeled as the bus system 4 in FIG. 11.

The memory 3 in the embodiment of the present disclosure is configured to store various types of data to support operations of the ultrasonic device. Examples of the data include any computer program executable on the ultrasonic device.

It can be understood that the memory 3 may be a transitory memory or a non-transitory memory, or may include both the transitory memory and the non-transitory memory. The non-transitory memory may be a read-only memory (ROM), a programmable read-only memory (PROM), an erasable programmable read-only memory (EPROM), an electrically erasable programmable read-only memory (EEPROM), a ferromagnetic random-access memory (FRAM), a flash memory, a magnetic surface storage, an optical disk, or a compact disc read-only memory (CD-ROM). The magnetic surface storage may be a disk storage or a tape storage. The volatile memory may be a random-access memory (RAM) serving as an external cache memory. By way of illustration, but not limitation, many forms of RAM are available, such as a static random access memory (SRAM), a synchronous static random access memory (SSRAM), a dynamic random access memory (DRAM), a synchronous dynamic random access memory (SDRAM), a double data rate synchronous dynamic random access memory (DDRSDRAM), an enhanced synchronous dynamic random access memory (ESDRAM), a SyncLink dynamic random access memory (SLDRAM), and a direct rambus random access memory (DRRAM). The memory 3 described in the embodiments of the present disclosure is intended to include, but not limited to, these and any other suitable types of memory.

The method disclosed in the above embodiments of the present disclosure may be applied to the processor 2 or implemented by the processor 2. The processor 2 may be an integrated circuit chip having a signal processing capability. During an implementation process, a step of the above method may be completed by an integrated logic circuit in a form of hardware in the processor 2 or through software instructions. The processor 2 may be a general-purpose processor, a DSP, or other programmable logic devices, a discrete gate or transistor logic device, a discrete hardware component, or the like. The processor 2 may implement or execute the method, steps and logical block diagrams disclosed in the embodiments of the present disclosure. The general-purpose processor may be a microprocessor or any conventional processor. The steps of the method disclosed in conjunction with the embodiments of the present disclosure may be directly embodied and executed by a hardware decoding processor, or be executed by a combination of hardware and a software module in the decoding processor. The software module may be provided in a storage medium. The storage medium is located in the memory 3. The processor 2 reads a program in the memory 3 and completes the foregoing method in combination with hardware.

The processor 2, when executes the program, implements the method in the embodiments of the present disclosure. For sake of simplicity, the method is not repeated here.

In an exemplary embodiment, a storage medium, that is, a computer storage medium, is further provided in an embodiment of the present disclosure. The storage medium is a computer-readable storage medium, such as the memory 3 that stores a computer program. The computer program may be executed by the processor 2 to implement the method described in the previous embodiments. The computer-readable storage medium may be, for example, an FRAM, an ROM, a PROM, an EPROM, an EEPROM, a Flash Memory, a magnetic surface memory, an optical disk, or a CD-ROM.

Those of ordinary skills in the art can understand that all or part of the steps for implementing the above method embodiments may be completed through program instructions and related hardware. The program may be stored in a computer-readable storage medium. The program, when executed, performs steps including the above method embodiments. The storage medium includes a removable storage device, an ROM, an RAM, a magnetic disk, an optical disk, or other media capable of storing program codes.

Alternatively, in a case that the integrated unit mentioned above in the present disclosure is implemented in a form of a software functional module and sold or used as an independent product, the integrated unit may be stored in a computer-readable storage medium. Based on such understanding, part of the technical solutions of the embodiments of the present disclosure which are essential or contribute to the conventional technology may be embodied in a form of a software product. The computer software product is stored in a storage medium and includes instructions for causing an ultrasonic device (which may be a personal computer, a server, a network device, or the like) to perform all or part of the method described in the embodiments of the present disclosure. The aforementioned storage medium includes a mobile storage device, a ROM, a RAM, a magnetic disk, an optical disk, or other media capable of storing program codes.

Described above are only some specific embodiments of the present disclosure, and the protection scope of the present disclosure is not limited thereto. Any changes or alternations that can be easily envisaged by those familiar with the art within the technical scope disclosed in the present disclosure shall fall within the protection scope of the present disclosure. Therefore, the protection scope of the present disclosure shall be in accordance with the protection scope of the claims.

1. A method for ultrasonic contrast imaging, comprising:
determining a plurality of transmission frequencies within an effective bandwidth range of an ultrasonic probe, and generating a pulse sequence comprising a plurality of ultrasonic signal groups based on the plurality of transmission frequencies, wherein each of the ultrasonic signal groups comprises a plurality of ultrasonic signals, and each of the ultrasonic signals comprises at least two periods of ultrasonic sub-signals having different transmission frequencies, the plurality of ultrasonic signals in a same ultrasonic signal group have a same transmission frequency in a corresponding period, and the plurality of ultrasonic signals in different ultrasonic signal groups have different transmission frequencies in the corresponding period;
transmitting the plurality of ultrasonic signals in the plurality of ultrasonic signal groups in the pulse sequence sequentially, and receiving echo signals generated by contrast agent microbubbles in response to the transmitted ultrasonic signals, wherein the echo signals comprise fundamental signals and harmonic signals corresponding to different imaging frequencies;
processing the echo signals to obtain a plurality of contrast signals corresponding to different imaging frequencies, and generating contrast images based on the contrast signals; and
fusing the contrast images to obtain target contrast images.

2. The method for ultrasonic contrast imaging according to claim 1, wherein the determining the plurality of transmission frequencies within the effective bandwidth range of the ultrasonic probe, and generating the pulse sequence comprising the plurality of ultrasonic signal groups based on the transmission frequencies comprises:
determining a reference transmission frequency and a target coefficient, determining a product of the reference transmission frequency and the target coefficient as a first transmission frequency, determining twice the first transmission frequency as a second transmission frequency, determining three times the first transmission frequency as a third transmission frequency, and determining four times the first transmission frequency as a fourth transmission frequency, wherein the first transmission frequency, the second transmission frequency, the third transmission frequency and the fourth transmission frequency are all within the effective bandwidth range of the ultrasonic probe; and
generating the pulse sequence comprising a first ultrasonic signal group and a second ultrasonic signal group, wherein the first ultrasonic signal group is generated based on the first transmission frequency and the second transmission frequency, and the second ultrasonic signal group is generated based on the third transmission frequency and the fourth transmission frequency.

3. The method for ultrasonic contrast imaging according to claim 1, wherein the determining the plurality of transmission frequencies within the effective bandwidth range of the ultrasonic probe, and generating the pulse sequence comprising the plurality of ultrasonic signal groups based on the transmission frequencies comprises:
determining a reference transmission frequency and a target coefficient, determining a product of the reference transmission frequency and the target coefficient as a first transmission frequency, determining twice the first transmission frequency as a second transmission frequency, determining three times the first transmission frequency as a third transmission frequency, determining four times the first transmission frequency as a fourth transmission frequency, and determining a transmission frequency adjacent to the second transmission as a fifth transmission frequency, wherein the second transmission frequency, the third transmission frequency, the fourth transmission frequency and the fifth transmission frequency are all within the effective bandwidth range of the ultrasonic probe; and
generating the pulse sequence comprising a first ultrasonic signal group and a second ultrasonic signal group, wherein the first ultrasonic signal group is generated based on the fifth transmission frequency and the second transmission frequency, and the second ultrasonic signal group is generated based on the third transmission frequency and the fourth transmission frequency.

4. The method for ultrasonic contrast imaging according to claim 1, wherein the transmitting the multiple ultrasonic signals in the multiple ultrasonic signal groups in the pulse sequence sequentially comprises:
determining different voltage amplitudes for ultrasonic sub-signals in the corresponding period in the multiple ultrasonic signals of the same ultrasonic signal group;
determining a same number of array elements of the transmitting aperture for ultrasonic sub-signals in the multiple ultrasonic signals of the same ultrasonic signal group; and
transmitting the multiple ultrasonic signals in the multiple ultrasonic signal groups in the pulse sequence sequentially, based on the transmission frequencies, the voltage amplitudes and the numbers of array elements of the transmitting aperture for different ultrasonic sub-signals in the multiple ultrasonic signals.

5. The method for ultrasonic contrast imaging according to claim 4, wherein each of the ultrasonic signal groups comprises a first ultrasonic signal and a second ultrasonic signal, and the determining different voltage amplitudes for ultrasonic sub-signals in the corresponding period in the multiple ultrasonic signals of the same ultrasonic signal group comprises:
determining a first voltage amplitude for the ultrasonic sub-signal in the first ultrasonic signal, and determining a second voltage amplitude for the ultrasonic sub-signal in the second ultrasonic signal, wherein the second voltage amplitude is a preset multiple of the first voltage amplitude.

6. The method for ultrasonic contrast imaging according to claim 4, wherein each of the ultrasonic signal groups comprises a first ultrasonic signal, a second ultrasonic signal and a third ultrasonic signal, and the determining different voltage amplitudes for ultrasonic sub-signals in the corresponding period in the multiple ultrasonic signals of the same ultrasonic signal group comprises:
determining a third voltage amplitude for the ultrasonic sub-signal in the first ultrasonic signal and the ultrasonic sub-signal in the third ultrasonic signal, and determining a fourth voltage amplitude for the ultrasonic sub-signal in the second ultrasonic signal, wherein the fourth voltage amplitude is a preset multiple of the third voltage amplitude.

7. The method for ultrasonic contrast imaging according to claim 1, wherein the transmitting the multiple ultrasonic signals in the multiple ultrasonic signal groups in the pulse sequence sequentially comprises:
determining a same voltage amplitude for the ultrasonic sub-signals in the corresponding period in the multiple ultrasonic signals of the same ultrasonic signal group;
determining different numbers of array elements of the transmitting aperture for the ultrasonic sub-signals in the multiple ultrasonic signals of the same ultrasonic signal group; and
transmitting the multiple ultrasonic signals in the multiple ultrasonic signal groups in the pulse sequence sequentially, based on the transmission frequencies, the voltage amplitudes and the numbers of array elements of the transmitting aperture for different ultrasonic sub-signals in the multiple ultrasonic signals.

8. The method for ultrasonic contrast imaging according to claim 7, wherein each of the ultrasonic signal groups comprises a first ultrasonic signal and a second ultrasonic signal, and the determining different numbers of array elements of the transmitting aperture for the ultrasonic sub-signals in the multiple ultrasonic signals of the same ultrasonic signal group comprises:
determining a first number of array elements of the transmitting aperture for the ultrasonic sub-signals in the first ultrasonic signal, and determining a second number of array elements of the transmitting aperture for the ultrasonic sub-signals in the second ultrasonic signal, wherein the second number of array elements of the transmitting aperture is a preset multiple of the first number of array elements of the transmitting aperture.

9. The method for ultrasonic contrast imaging according to claim 8, wherein the second number of array elements of the transmitting aperture is twice the first number of array elements of the transmitting aperture.

10. The method for ultrasonic contrast imaging according to claim 7, wherein each of the ultrasonic signal groups comprises a first ultrasonic signal, a second ultrasonic signal and a third ultrasonic signal, and the determining different numbers of array elements of the transmitting aperture for the ultrasonic sub-signals in the multiple ultrasonic signals of the same ultrasonic signal group comprises:
determining the transmitting aperture of the ultrasonic sub-signal in the first ultrasonic signal as one of an odd aperture and an even aperture, determining the transmitting aperture of the ultrasonic sub-signal in the second ultrasonic signal as a full aperture, and determining the transmitting aperture of the ultrasonic sub-signal in the third ultrasonic signal as the other one of the odd aperture and the even aperture.

11. The method for ultrasonic contrast imaging according to claim 1, wherein the processing the echo signals to obtain a plurality of contrast signals corresponding to different imaging frequencies comprises:
performing weighted processing on the echo signals of the multiple ultrasonic signals in each of the ultrasonic signal groups, based on a voltage amplitude relationship and a phase relationship among the multiple ultrasonic signals in each of the ultrasonic signal groups, to obtain the contrast signals corresponding to each of the ultrasonic signal groups; and
demodulating the contrast signals corresponding to each of the ultrasonic signal groups by using demodulation circuits corresponding to the different imaging frequencies, to obtain the contrast signals corresponding to the different imaging frequencies.

12. The method for ultrasonic contrast imaging according to claim 11, wherein the performing weighted processing on the echo signals of the multiple ultrasonic signals in each of the ultrasonic signal groups, based on the voltage amplitude relationship and the phase relationship among the multiple ultrasonic signals in each of the ultrasonic signal groups, to obtain the contrast signals corresponding to each of the ultrasonic signal groups, comprises:
determining weighting coefficients of the echo signals, based on the voltage amplitude relationship among the multiple ultrasonic signals in each of the ultrasonic signal groups;
determining an operational relationship of the echo signals based on the phase relationship among the multiple ultrasonic signals in each of the ultrasonic signal groups, wherein the operational relationship is determined as a summation in response to the phase relationship being out of phase, and the operational relationship is determined as a subtraction in response to the phase relationship being in phase;
performing weighted processing on the echo signals of the multiple ultrasonic signals in each of the ultrasonic signal groups based on the weighting coefficients and the operational relationship, to obtain the contrast signals corresponding to each of the ultrasonic signal groups.

13. The method for ultrasonic contrast imaging according to claim 1, wherein the transmitting the multiple ultrasonic signals in the multiple ultrasonic signal groups in the pulse sequence sequentially, and receiving the echo signals generated by the contrast agent microbubbles in response to the transmitted ultrasonic signals, comprises:
transmitting the multiple ultrasonic signals in the multiple ultrasonic signal groups in the pulse sequence sequentially, wherein in response to the transmitted ultrasonic signals, the contrast agent microbubbles generate fundamental signals corresponding to different first imaging frequencies, generate a multi-harmonic signal corresponding to a second imaging frequency based on the fundamental signals corresponding to the first imaging frequencies, and generate a differential harmonic signal corresponding to a third imaging frequency based on the fundamental signals corresponding to the different first imaging frequencies, wherein the second imaging frequency is an integer multiple of the first imaging frequency, and the third imaging frequency is at least one of a difference between first imaging frequencies or a sum of first imaging frequencies; and
receiving the fundamental signals corresponding to the first imaging frequencies, the multi-harmonic signal corresponding to the second imaging frequency, and the differential harmonic signal corresponding to the third imaging frequency.

14. The method for ultrasonic contrast imaging according to claim 1, wherein the fusing the contrast images to obtain the target contrast images comprises:
determining weighting coefficients of contrast images corresponding to different imaging modes, and performing weighted fusion on the contrast images based on the weighting coefficients of the contrast images, to obtain the target contrast images corresponding to the different imaging modes.

15. The method for ultrasonic contrast imaging according to claim 14, wherein the imaging modes comprise a penetration mode and/or a resolution mode, the weighting coefficient of the contrast image corresponding to the penetration mode is negatively correlated with an imaging frequency corresponding to the contrast image, and the weighting coefficient of the contrast image corresponding to the resolution mode is positively correlated with the imaging frequency corresponding to the contrast image.

16. The method for ultrasonic contrast imaging according to claim 14, wherein the determining the weighting coefficients of the contrast images corresponding to different imaging modes comprises:
displaying, for one target imaging mode, an adjustment window corresponding to the target contrast image, wherein the adjustment window comprises adjustment areas for the contrast images; and
receiving an adjustment instruction on at least one of the adjustment areas and adjusting the weighting coefficient of the contrast image corresponding to the at least one of the adjustment areas based on the adjustment instruction, to obtain the weighting coefficients of the contrast images in the target imaging mode.

17. (canceled)

18. An ultrasonic imaging system, comprising an ultrasonic host, an ultrasonic probe, and a master computer, wherein the ultrasonic host is communicatively connected to the ultrasonic probe and the master computer; and
the ultrasonic host is configured to perform a method for ultrasonic contrast imaging, wherein the method comprises:
determining a plurality of transmission frequencies within an effective bandwidth range of an ultrasonic probe, and generating a pulse sequence comprising a plurality of ultrasonic signal groups based on the plurality of transmission frequencies, wherein each of the ultrasonic signal groups comprises a plurality of ultrasonic signals, and each of the ultrasonic signals comprises at least two periods of ultrasonic sub-signals having different transmission frequencies, the plurality of ultrasonic signals in a same ultrasonic signal group have a same transmission frequency in a corresponding period, and the plurality of ultrasonic signals in different ultrasonic signal groups have different transmission frequencies in the corresponding period;
transmitting the plurality of ultrasonic signals in the plurality of ultrasonic signal groups in the pulse sequence sequentially, and receiving echo signals generated by contrast agent microbubbles in response to the transmitted ultrasonic signals, wherein the echo signals comprise fundamental signals and harmonic signals corresponding to different imaging frequencies;
processing the echo signals to obtain a plurality of contrast signals corresponding to different imaging frequencies, and generating contrast images based on the contrast signals; and
fusing the contrast images to obtain target contrast images.

19. An ultrasonic device, comprising:
a memory, configured to store a computer program; and
a processor, configured to execute the computer program, to cause the ultrasonic device to:
determine a plurality of transmission frequencies within an effective bandwidth range of an ultrasonic probe, and generate a pulse sequence comprising a plurality of ultrasonic signal groups based on the plurality of transmission frequencies, wherein each of the ultrasonic signal groups comprises a plurality of ultrasonic signals, and each of the ultrasonic signals comprises at least two periods of ultrasonic sub-signals having different transmission frequencies, the plurality of ultrasonic signals in a same ultrasonic signal group have a same transmission frequency in a corresponding period, and the plurality of ultrasonic signals in different ultrasonic signal groups have different transmission frequencies in the corresponding period;
transmit the plurality of ultrasonic signals in the plurality of ultrasonic signal groups in the pulse sequence sequentially, and receive echo signals generated by contrast agent microbubbles in response to the transmitted ultrasonic signals, wherein the echo signals comprise fundamental signals and harmonic signals corresponding to different imaging frequencies;
process the echo signals to obtain a plurality of contrast signals corresponding to different imaging frequencies, and generate contrast images based on the contrast signals; and
fuse the contrast images to obtain target contrast images.

20. (canceled)

21. The ultrasonic device according to claim 19, wherein the processor is configured to execute the computer program to cause the ultrasonic device to:
determine a reference transmission frequency and a target coefficient, determine a product of the reference transmission frequency and the target coefficient as a first transmission frequency, determine twice the first transmission frequency as a second transmission frequency, determine three times the first transmission frequency as a third transmission frequency, and determine four times the first transmission frequency as a fourth transmission frequency, wherein the first transmission frequency, the second transmission frequency, the third transmission frequency and the fourth transmission frequency are all within the effective bandwidth range of the ultrasonic probe; and
generate the pulse sequence comprising a first ultrasonic signal group and a second ultrasonic signal group, wherein the first ultrasonic signal group is generated based on the first transmission frequency and the second transmission frequency, and the second ultrasonic signal group is generated based on the third transmission frequency and the fourth transmission frequency.

22. The ultrasonic device according to claim 19, wherein the processor is configured to execute the computer program to cause the ultrasonic device to:
determine a reference transmission frequency and a target coefficient, determine a product of the reference transmission frequency and the target coefficient as a first transmission frequency, determine twice the first transmission frequency as a second transmission frequency, determine three times the first transmission frequency as a third transmission frequency, determine four times the first transmission frequency as a fourth transmission frequency, and determine a transmission frequency adjacent to the second transmission as a fifth transmission frequency, wherein the second transmission frequency, the third transmission frequency, the fourth transmission frequency and the fifth transmission frequency are all within the effective bandwidth range of the ultrasonic probe; and
generate the pulse sequence comprising a first ultrasonic signal group and a second ultrasonic signal group, wherein the first ultrasonic signal group is generated based on the fifth transmission frequency and the second transmission frequency, and the second ultrasonic signal group is generated based on the third transmission frequency and the fourth transmission frequency.
